(19) United States
(12) Patent Application Publication
(10) Pub. No.: US 2025/0170278 A1
(43) Pub. Date: May 29, 2025

(54) Near-Infrared Dyes And Conjugates For Targeting Tumors

(71) Applicant: Lahjavida, LLC, Colorado Springs, CO (US)

(72) Inventors: Lyle D. Small, Colorado Springs, CO (US); Ruizheng Wang, Colorado Springs, CO (US); Shane M. Crippen, Colorado Springs, CO (US)

(73) Assignee: Lahjavida, LLC, Colorado Springs, CO (US)

(21) Appl. No.: 18/869,200

(22) PCT Filed: May 23, 2023
(86) PCT No.: PCT/US23/23173

§ 371 Date: Nov. 25, 2024

Related U.S. Application Data
(60) Provisional application No. 63/344,972, filed on May 23, 2022.

(51) Int. Cl. A61K 49/00; A61K 41/00

(52) U.S. Cl. CPC A61K 49/0032; A61K 41/0052

(57) ABSTRACT

The design, synthesis, and functionalization of a conjugate including a tumor-targeting near-infrared (NIR) dye and a therapeutic agent and/or a diagnostic agent, whereby the NIR dye can function to target the therapeutic agent and/or diagnostic agent to tumor cells.

This International Patent Cooperation Treaty patent application claims the benefit of U.S. Provisional Patent Application No. 63/344,972, filed May 23, 2022, hereby incorporated by reference herein.

### I. BACKGROUND

There remains a need for targeted cancer therapeutic agents with maximal efficacy in treating or killing tumor cells and minimal toxicity toward non-tumor or healthy cells. Additionally, there remains a need for a targeted cancer diagnostic agent.

### II. DISCLOSURE OF THE INVENTION

Generally, the present invention details the use of tumor-targeting near-infrared (NIR) fluorescent dyes in conjunction with a variety of therapeutic agents and/or diagnostic agents.

Following, a broad object of a particular embodiment of the invention can be to provide a conjugate including a tumor-targeting NIR dye and a therapeutic agent, whereby the dye functions to target and/or deliver the therapeutic agent to tumor cells.

Another broad object of a particular embodiment of the invention can be to provide a conjugate including a tumor-targeting NIR dye and a diagnostic agent, whereby the dye functions to target and/or deliver the diagnostic agent to tumor cells.

Another broad object of a particular embodiment of the invention can be to provide a method of killing tumor cells by targeting a conjugate including a tumor-targeting NIR dye and a therapeutic agent to said tumor cells.

Another broad object of a particular embodiment of the invention can be to provide a method of diagnosing the presence of tumor cells by targeting a conjugate including a tumor-targeting NIR dye and a diagnostic agent to said tumor cells, if present.

Naturally, further objects of the invention are disclosed throughout other areas of the specification, drawings, and claims.

### III. BRIEF DESCRIPTION OF THE DRAWINGS

FIG. 1 is an illustration of the NIR intensity mean per well over time for a lung cancer cell line (A549) and four normal cell lines (colon cells, kidney cells, liver cells, and HUVECs) following a 20 μM dose of the dye shown in Formula V.

FIG. 2 shows illustrative examples of zwitterions which may be useful with the present invention when attached to a tumor-targeting NIR dye and/or a nanoparticle.

FIG. 3 shows the hydration capabilities of zwitterions useful with the present invention having positively and negatively charged groups which can strongly associate with polar water molecules via ion-dipole interactions.

FIG. 4 shows an exemplary molecule which includes a negatively charged sulfonate group and a positively charged indole group, whereby the positive charge of the nitrogen is part of a resonance structure which delocalizes said positive charge.

FIG. 5A shows the experimental heating temperature profile of gold nanorods irradiated with an NIR laser at a wavelength of 808 nm and different power densities.

FIG. 5B shows the experimental heating temperature profile of gold nanorod conjugates irradiated with an NIR laser at a wavelength of 808 nm and different power densities.

FIG. 5C shows the theoretical heating temperature profile of gold nanorods irradiated with an NIR laser at a wavelength of 808 nm and different power densities.

FIG. 5D shows the theoretical heating temperature profile of gold nanorod conjugates irradiated with an NIR laser at a wavelength of 808 nm and different power densities.

FIG. 6 shows the experimental heating temperature profile of gold nanorods and gold nanorod conjugates irradiated with an LED light source.

FIG. 7 shows the UV-vis extinction spectra of gold nanorods (GNR, blue line) and gold nanorod conjugates with the dye of Formula II (GNRC, red line).

FIG. 8 shows the UV-vis extinction spectra of the dye of Formula II.

FIG. 9A shows the dynamic light scattering (DLS) size distribution of gold nanorods.

FIG. 9B shows the dynamic light scattering (DLS) size distribution of gold nanorod conjugates with the dye of Formula II.

FIG. 10A illustrates a particular embodiment of the present conjugate which includes a tumor-targeting NIR dye attached to a zwitterionic nanoparticle.

FIG. 10B illustrates a particular embodiment of the present conjugate which includes a zwitterionic tumor-targeting NIR dye attached to a zwitterionic nanoparticle.

FIG. 11 shows a particular embodiment of the present conjugate including DOX conjugated to a tumor-targeting NIR dye via a cleavable linker with pH sensitivity having an acid-labile hydrazone bond.

FIG. 12A shows a particular embodiment of the present conjugate including a PEG linker and SN-38 as the chemotherapeutic agent.

FIG. 12B shows a particular embodiment of the present conjugate including a PEG linker and SN-38 as the chemotherapeutic agent.

FIG. 12C shows a particular embodiment of the present conjugate including a PEG linker and DOX as the chemotherapeutic agent.

FIG. 12D shows a particular embodiment of the present conjugate including a PEG linker and DOX as the chemotherapeutic agent.

FIG. 13 shows a particular embodiment of the present conjugate having a linker including a VC dipeptide which can be cleaved by cathepsin B to release the conjugate portion including the chemotherapeutic agent (SN-38) and a self-immolative PABC spacer. Subsequently, PABC can undergo spontaneous 1,6-elimination in an acidic environment to release carbon dioxide, para azaquinone methide, and SN-38.

FIG. 14A shows a particular embodiment of the present conjugate made using click chemistry and including SN-38 as the chemotherapeutic agent.

FIG. 14B shows a particular embodiment of the present conjugate made using click chemistry and including SN-38 as the chemotherapeutic agent.

FIG. 14C shows a particular embodiment of the present conjugate made using click chemistry and including DOX as the chemotherapeutic agent.

FIG. 14D shows a particular embodiment of the present conjugate made using click chemistry and including DOX as the chemotherapeutic agent.

### IV. MODE(S) FOR CARRYING OUT THE INVENTION

The present invention may provide an effective and versatile delivery method which combines tumor-targeting near-infrared (NIR) fluorescent dyes with various cancer therapeutic agents and/or cancer diagnostic agents to create conjugates for (i) targeting tumor cells and/or (ii) killing tumor cells and/or (iii) imaging tumor cells and/or (iv) theranostics, whereby the NIR dye functions to direct the conjugate to the target tumor cell population; this may be in contrast to the non-specific delivery of conventional anticancer agents which can cause significant adverse side effects. In addition to targeting tumor cells, the present NIR dye can absorb NIR light and emit in the same region with appreciable brightness, whereby such a property may be extremely desirable considering the noninvasive nature of NIR light and its tissue penetration, which can be orders of magnitude greater than that for ultraviolet or visible light.

As to particular embodiments, the tumor-targeting NIR dye can be a cyanine dye, whereby chemically, cyanine dyes include two nitrogen atoms joined by a polymethine chain.

As to particular embodiments, the tumor-targeting NIR dye can be a heptamethine cyanine dye.

As to particular embodiments, the tumor-targeting NIR dye can have Formula I as follows:

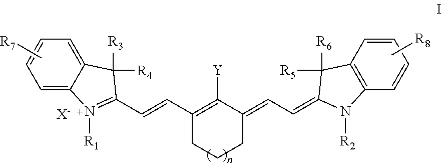

wherein:
R₁ to R₈ can each be independently an unsubstituted or substituted C₁ to C₁₀ alkyl group, an unsubstituted or substituted aryl group, an unsubstituted or substituted C₁ to C₁₀ alkoxyl group, an alkylsulfonate, an alkyl carboxylic acid group, or an alkylamino group;
X can be Br⁻, Cl⁻, I⁻, perchlorate (ClO₄⁻), tosylate PTS⁻, or absent if another covalently linked anion moiety is present in the dye molecule;
Y can be Cl, substituted C, O, S, or N; and
n can be 0 or 1.

As to particular embodiments, one or more of R₁ to R₈ can be functionalized via various linkers with a zwitterion(s), a small molecule chemotherapy drug(s), a photodynamic therapy (PDT) agent(s), or a hyperthermia agent(s), such as gold nanoparticles, iron oxide nanoparticles, or the like.

As to particular embodiments, regarding Y, the substituted C, O, S, or N can be functionalized via various linkers with a zwitterion(s), a small molecule chemotherapy drug(s), a photodynamic therapy (PDT) agent(s), or a hyperthermia agent(s), such as gold nanoparticles, iron oxide nanoparticles, or the like.

As to particular embodiments, the linker can be heterobifunctional, thus reacting with both the tumor-targeting NTR dye and the therapeutic or diagnostic agent to form a conjugate, whereby as only a few illustrative examples, the linker may be a polyethylene glycol (PEG), a polymer, a peptide, DNA, a silica nanoparticle, a shell structure (such as iron-oxide or silica), an amide bond, an ester bond, an amine, an alcohol, a phenol, a thio, a phenyl or derivatives thereof, or the like.

As to particular embodiments, the linker can be cleavable.

The present tumor-targeting NTR dyes can be symmetrical or asymmetrical, whereby exemplary dyes are shown in Formulas II, III, IV, and V.

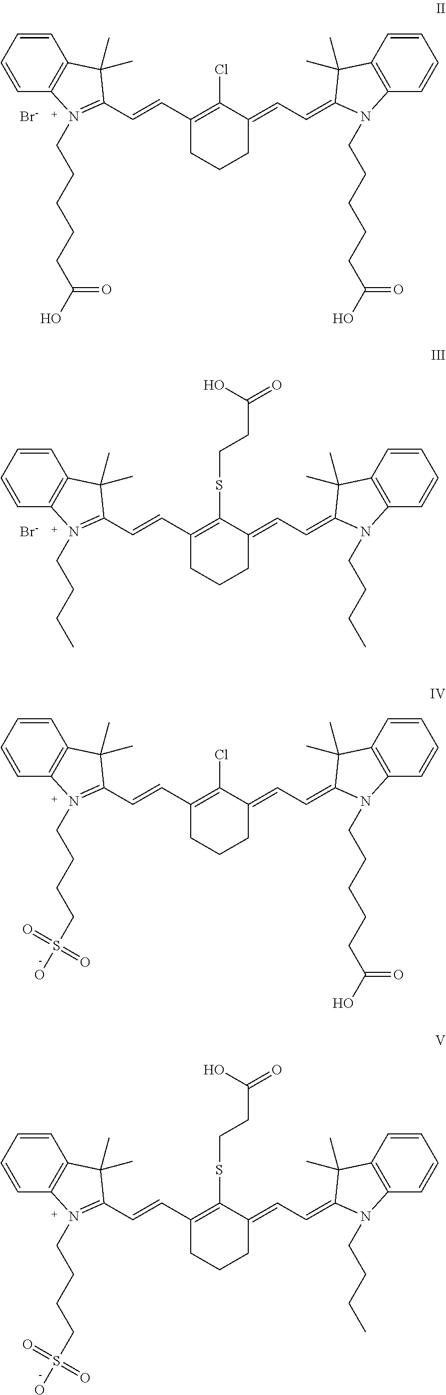

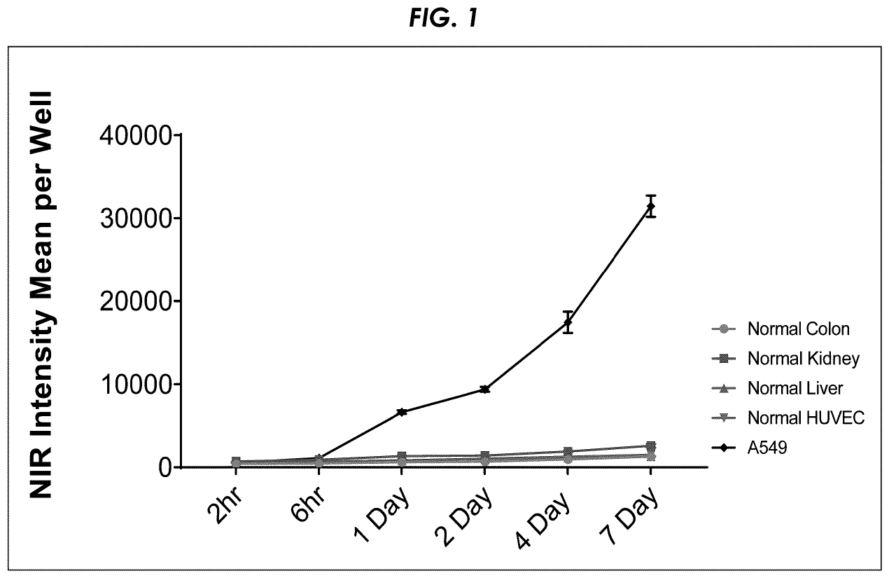

To illustrate the tumor-targeting ability of a particular embodiment of a tumor-targeting NIR dye and specifically, the dye shown in Formula V, cells were dosed with 20 μM of said dye (absorbance of ˜780 nm, emission of ˜808 nm) and the NIR intensity mean per well over time correlating to dye uptake is shown in FIG. 1 for a lung cancer cell line (A549) versus four normal cell lines (colon cells, kidney cells, liver cells, and human umbilical vein endothelial cells (HUVEC)), whereby significant dye uptake is evident in only the cancer cell line.

As to particular embodiments, the tumor-targeting NIR dye can be modified to have zwitterionic functionality, whereby such a modification can include the attachment of one or more zwitterions to the dye, each said zwitterion functioning as a polyelectrolyte or dipolar ion which has both positively and negatively charged groups, yet can be overall neutral in charge. Additionally, zwitterions can have a high hydration capacity and correspondingly can be highly hydrated via electrostatic interactions of their positively and negatively charged groups with polar water molecules, whereby the resultant tightly bound water layer forms a dense and stable hydration shell or solvation shell which can provide a physical and energetic barrier that can prevent undesired adsorption, such as but not limited to protein adsorption; thus, zwitterions can have effective antifouling properties.

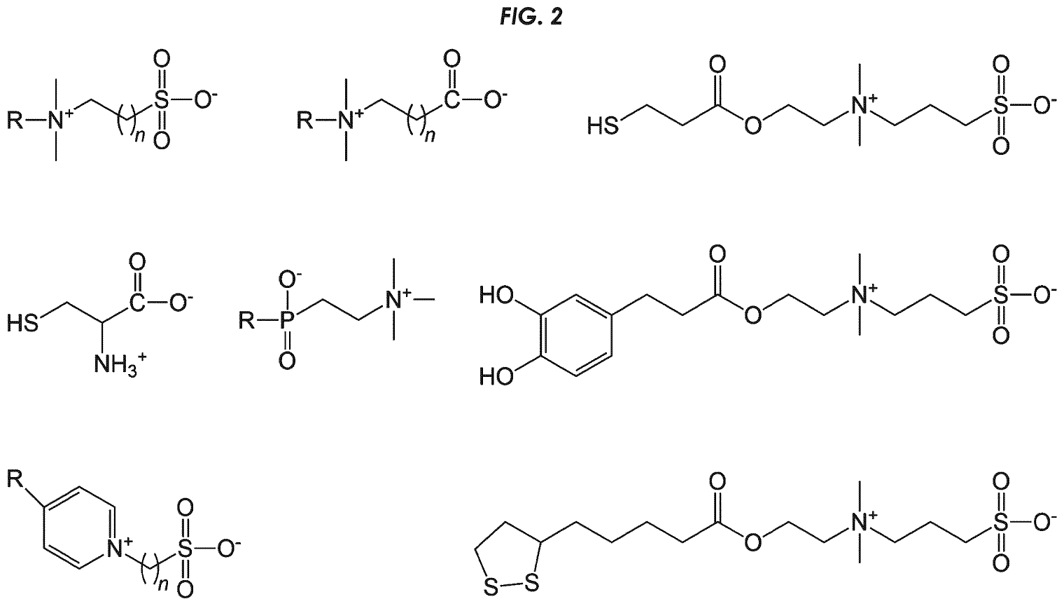

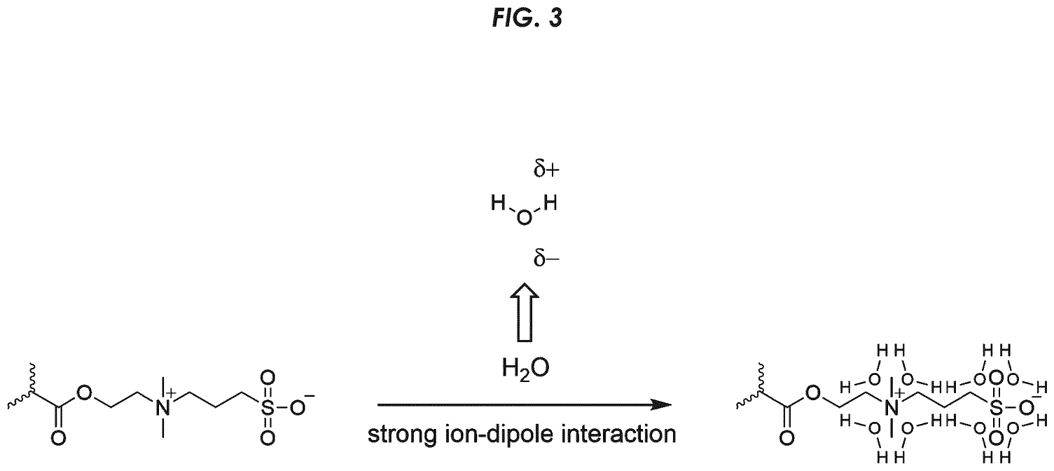

As to particular embodiments, illustrative examples of zwitterions which may be useful with the present invention when attached to a tumor-targeting NIR dye include sulfobetaine, carboxybetaine, phosphorylcholine, and those shown in FIG. 2. In line with the above, the zwitterions shown in FIG. 2 can be overall neutrally charged and have remarkable hydration capabilities via strong ion-dipole interactions of their positively and negatively charged groups with polar water molecules, as shown in FIG. 3.

To impart zwitterionic functionality to the tumor-targeting NIR dye, a zwitterion can be covalently or electrostatically attached or bound to the dye via the central ring (such as the central cyclopentyl or cyclohexyl ring), via the nitrogen of an indole moiety, via a geminal position, or via an aromatic ring. As to particular embodiments, one or more of R₁ to R₈ or Y as shown in Formula I can be used as a linking site to attach or bind a zwitterion to the tumor-targeting NTR dye via various linkers to form the present conjugate.

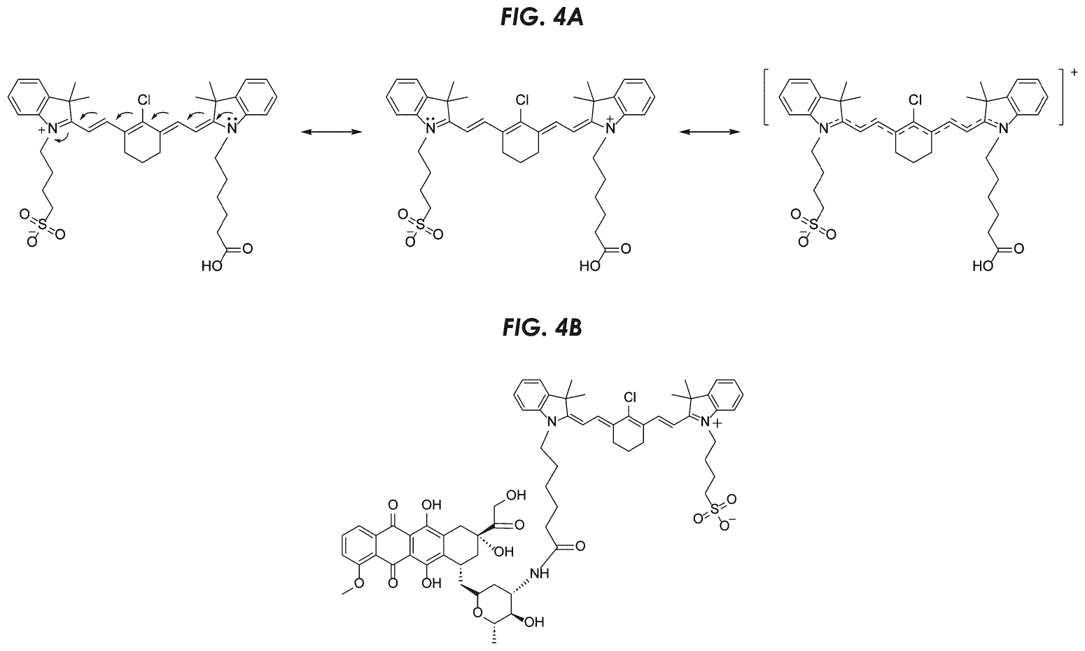

Significantly, while some molecules may be polyelectrolytes with both positively and negatively charged groups and have an overall neutral charge, due to one or more charged atoms being part of a resonance structure, these molecules may not have the high hydration capabilities needed for the present invention. To elaborate, if a charged atom is part of a resonance structure, that charge is delocalized or spread over multiple atoms as opposed to being associated with a single atom. Following, such a “diluted” charge loses its hydration capabilities via electrostatic interactions with polar water molecules and correspondingly, may not be useful with the present invention. As but one illustrative example, the molecule shown in FIG. 4A includes a negatively charged sulfonate group and a positively charged indole group; the positive charge of the nitrogen is part of a resonance structure involving a total of eleven atoms. Correspondingly, this positive charge is delocalized and thus, does not have high hydration capabilities via electrostatic interactions with polar water molecules. Following, such a molecule may not be useful with the present invention.

As another illustrative example, the molecule shown in FIG. 4B also includes a negatively charged sulfonate group and a positively charged indole group. Akin to the above, the positive charge of the nitrogen is part of a resonance structure involving a total of eleven atoms. Correspondingly, this positive charge is delocalized and thus, does not have high hydration capabilities via electrostatic interactions with polar water molecules. Following, such a molecule may not be useful with the present invention.

Similarly, the charged groups in the two NIR dyes shown above in Formulas IV and V may not have high hydration capabilities via electrostatic interactions with polar water molecules as a result of their resonance structures.

In view of the above, as to particular embodiments, zwitterions useful with the present invention can include charged groups in which the charge is associated with a single atom as opposed to being delocalized over multiple atoms, as this property imparts the desired high hydration capabilities of the zwitterion via electrostatic interactions with polar water molecules to form the hydration shell which can provide a physical and energetic barrier that prevents undesired adsorption.

As to particular embodiments, the zwitterionic tumor-targeting NIR dye can have Formula VI as follows:

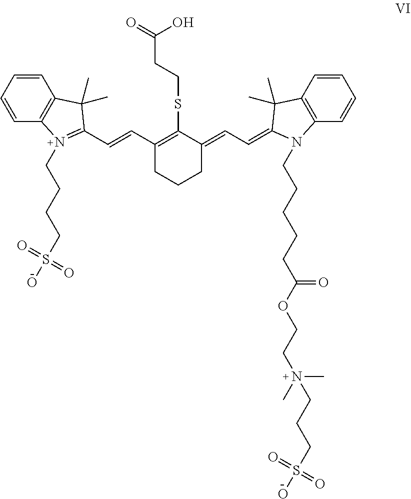

As to particular embodiments, the zwitterionic tumor-targeting NIR dye can have Formula VII as follows:

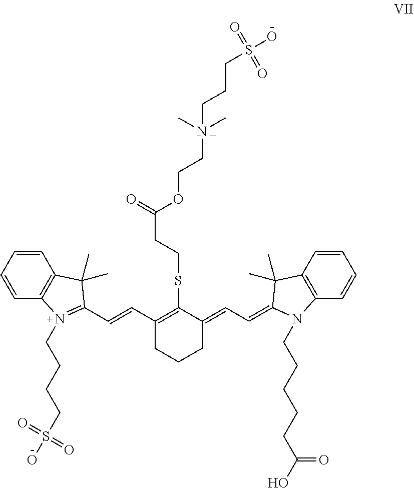

As to particular embodiments, the zwitterionic tumor-targeting NIR dye can have Formula VIII as follows:

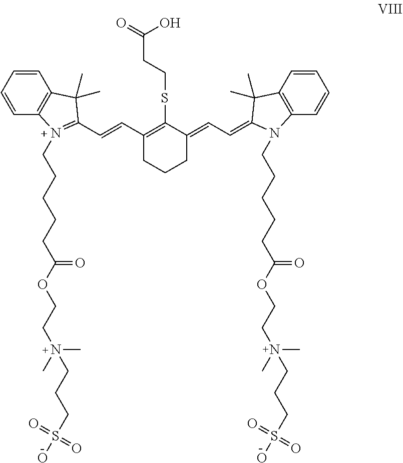

As to particular embodiments, the zwitterionic tumor-targeting NIR dye can have Formula IX as follows:

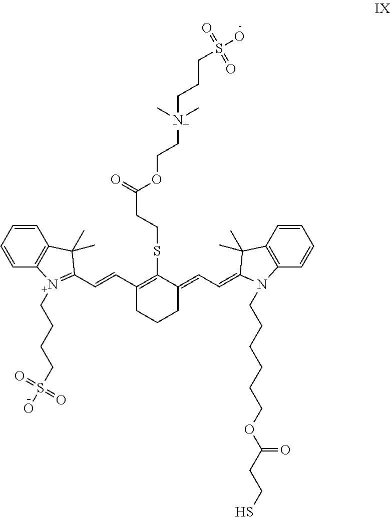 [SMILES: CC1(C)C(/C=C/C2=C(SCCC(=O)OCC[N+](C)(C)CCCS(=O)(=O)O)C(=C/C=C3/N(CCCCCCOC(=O)CCS)c4ccccc4C3(C)C)/CCC2)=[N+](CCCCS(=O)(=O)O)c2ccccc21]

As to particular embodiments, the zwitterionic tumor-targeting NIR dye can have Formula X as follows:

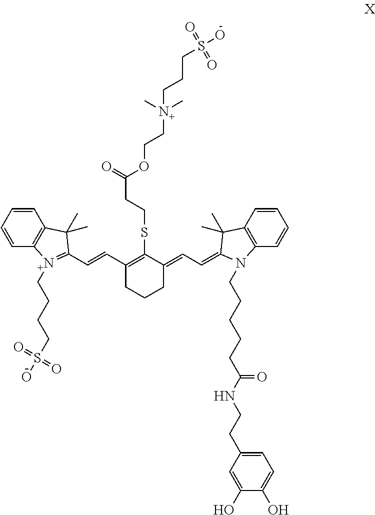 [SMILES: CC1(C)C(/C=C/C2=C(SCCC(=O)OCC[N+](C)(C)CCCS(=O)(=O)O)C(=C/C=C3/N(CCCCCC(=O)NCCc4ccc(O)c(O)c4)c4ccccc4C3(C)C)/CCC2)=[N+](CCCCS(=O)(=O)O)c2ccccc21]

As stated above, the present invention may combine a tumor-targeting NIR fluorescent dye with various agents to create conjugates for the treatment of different cancers, whereby these conjugates can be delivered directly to the target tumor cell population. To form such conjugates, a therapeutic or diagnostic agent can be covalently or electrostatically attached or bound to the tumor-targeting NIR dye, for example via the central ring (such as the central cyclopentyl or cyclohexyl ring), via the nitrogen of an indole moiety, via a geminal position, or via an aromatic ring. As to particular embodiments, one or more of R₁ to R₈ or Y as shown in Formula I can be used as a linking site to attach or bind the therapeutic or diagnostic agent to the tumor-targeting NIR dye via various linkers to form the present conjugate.

As to particular embodiments, a conjugate including a tumor-targeting NIR dye and a therapeutic or diagnostic agent can have Formula XI as follows:

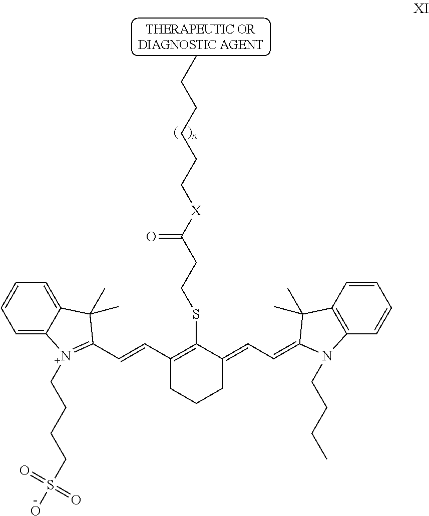

wherein X can be NH or O.

As to particular embodiments, a conjugate including a tumor-targeting NIR dye and a therapeutic or diagnostic agent can have Formula XII as follows:

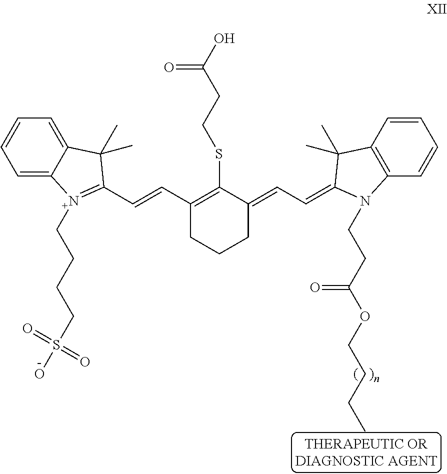

As to particular embodiments, a conjugate including a zwitterionic tumor-targeting NIR dye and a therapeutic or diagnostic agent can have Formula XIII as follows:

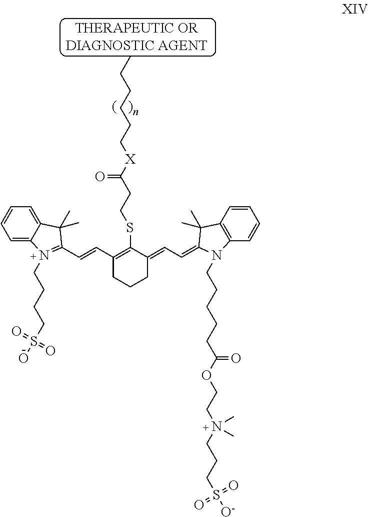

wherein X can be NH or O.

As to particular embodiments, a conjugate including a zwitterionic tumor-targeting NIR dye and a therapeutic or diagnostic agent can have Formula XIV as follows:

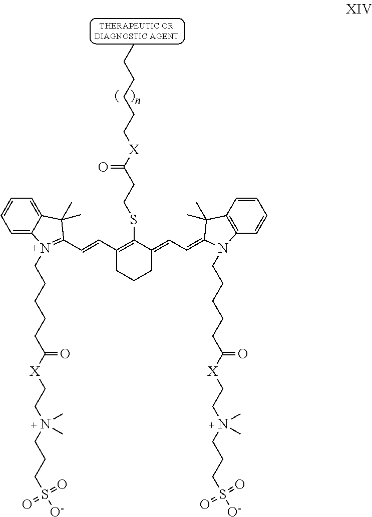

Regarding therapeutic agents, as to particular embodiments, a tumor-targeting NIR dye can be coupled to (for example via conjugation) a hyperthermia agent via various linkers for use in hyperthermia (also referred to as thermal therapy or thermotherapy), a type of cancer treatment in which body tissue can be exposed to high temperatures (heat) to damage and/or kill cancer cells or to make cancer cells more sensitive to the effects of radiation and/or certain anticancer drugs. Following, a hyperthermia agent can generate heat upon exposure to energy, such as radio waves, microwaves, infrared light, alternating magnetic fields, or ultrasound waves. As to particular embodiments, a hyperthermia agent can be a photothermal therapy agent, which can induce cancer cell death or sensitization by heat generated in tumor tissue exposed to NIR light.

As to particular embodiments in which the present conjugate includes a hyperthermia agent, the NIR dye can function to direct the conjugate including the hyperthermia agent to the target tumor cell population; this may be in contrast to the non-specific delivery of conventional hyperthermia agents which can cause significant adverse side effects when the recipient is exposed to the corresponding irradiation.

As an illustrative example, a hyperthermia agent can comprise a metal or semiconducting nanoparticle (for example gold, silver, iron oxide, carbon, or the like) which can heat in an electromagnetic field, i.e. a radiofrequency (RF) or NIR field.

As to particular embodiments, a hyperthermia agent can comprise a metal nanoparticle configured as a gold nanoparticle (AuNP), such as a gold nanosphere, a gold nanorod, a gold nanoshell, a gold nanomatryoshka, a gold nanobox, a gold nanocage, a gold nanostar, a silica-coated gold nanorod, a gold nanodimer, or the like.

As to particular embodiments, a conjugate including a tumor-targeting NIR dye and a gold nanoparticle which may be useful with the present invention can have Formula XV as follows:

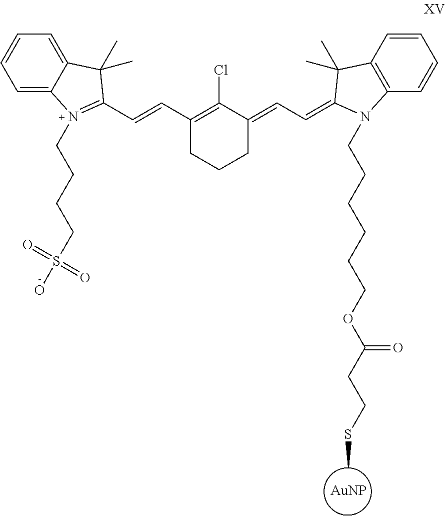

Of note, a tumor-targeting NIR dye having an —SH functionality can directly conjugate to a gold nanoparticle via a relatively strong S—AuNP bond (such as the conjugate shown in Formula XV).

As stated above, a conjugate comprising a gold nanoparticle can heat in an electromagnetic field, i.e. a RF or NIR field, and function as a hyperthermia agent.

Following, as to particular embodiments, a method of using the present conjugate including (i) a tumor-targeting NIR dye attached to a gold nanoparticle or (ii) a zwitterionic tumor-targeting NIR dye attached to a gold nanoparticle or (iii) a tumor-targeting NIR dye attached to a zwitterionic coated gold nanoparticle or (iv) a zwitterionic tumor-targeting NIR dye attached to a zwitterionic coated gold nanoparticle can be to administer the conjugate to a patient (such as via injection), whereby following administration, the NIR dye can direct the conjugate to the target tumor cell population. Subsequently, an RF field can be generated, whereby exposure to the RF field can result in heating of the gold nanoparticles and consequent induction of localized hyperthermia in the tumor cells.

As to other particular embodiments, a method of using the present conjugate including (i) a tumor-targeting NIR dye attached to a gold nanoparticle or (ii) a zwitterionic tumor-targeting NIR dye attached to a gold nanoparticle or (iii) a tumor-targeting NIR dye attached to a zwitterionic coated gold nanoparticle or (iv) a zwitterionic tumor-targeting NIR dye attached to a zwitterionic coated gold nanoparticle can be to administer the conjugate to a patient (such as via injection), whereby following administration, the NIR dye can direct the conjugate to the target tumor cell population. Subsequently, an NIR field can be generated (for example via laser irradiation or light-emitting diode (LED) irradiation), whereby upon exposure to the NIR field, the gold nanoparticles can convert absorbed light into heat and induce localized hyperthermia in the tumor cells, with minimal damage to intervening and surrounding normal tissue. In particular, gold nanoparticles and specifically, gold nanorods, may perform as strong NIR light absorbers, whereby such therapy can exploit the naturally occurring deficit of NIR-absorbing chromophores in most tissue, thereby permitting transmission of NIR light through tissue with scattering-limited attenuation and minimal heating. Light within the NIR spectral region has been shown to penetrate tissue with high spatial precision at depths beyond 1 cm with no observable damage to the intervening tissue, consequently allowing noninvasive delivery of heat to a tissue volume by using extracorporeal, low-power irradiation to selectively induce hyperthermia in tumor cells targeted with gold nanoparticles/gold nanorods.

Notably, the present conjugate including (i) a tumor-targeting NTR dye attached to a gold nanoparticle (and in particular, a gold nanorod) or (ii) a zwitterionic tumor-targeting NTR dye attached to a gold nanoparticle (and in particular, a gold nanorod) or (iii) a tumor-targeting NIR dye attached to a zwitterionic coated gold nanoparticle (and in particular, a gold nanorod) or (iv) a zwitterionic tumor-targeting NIR dye attached to a zwitterionic coated gold nanoparticle (and in particular, a gold nanorod) may, upon irradiation within the wavelength range of about 700 nanometers to about 1,500 nanometers (such as via a laser or LED), produce synergistic heating of the conjugate, meaning the conjugate may produce more heat than either the tumor-targeting NIR dye or the gold nanorod alone or a mixture of the two (not covalently bound together) when irradiated with NIR light; thus, the conjugate may have a greater light-to-heat transduction efficiency.

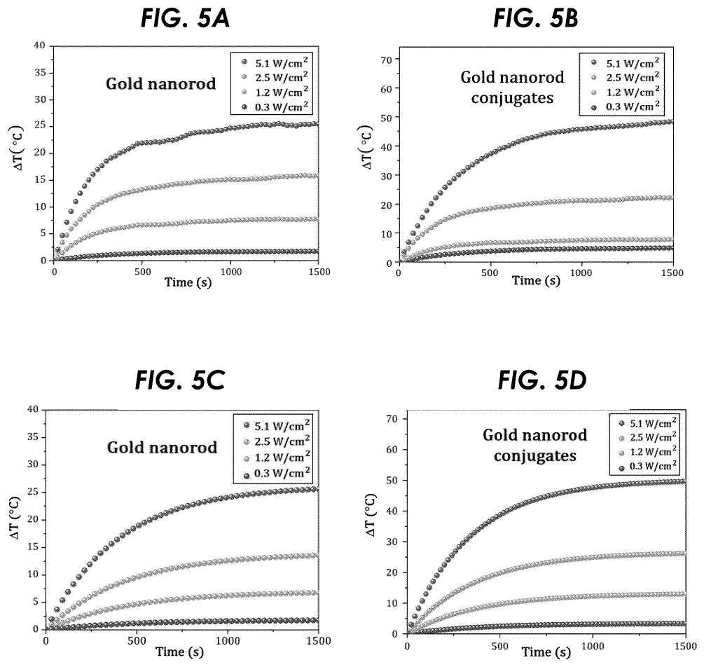

FIGS. 5A through 5D show experimental and theoretical heating temperature profiles of gold nanorods and gold nanorod conjugates irradiated with an NIR laser at 808 nm wavelength and different power densities (fluence). Specifically, FIGS. 5A and 5C show the experimental and theoretical temperature profiles over time for gold nanorods, respectively, and FIGS. 5B and 5D show the experimental and theoretical temperature profiles over time for gold nanorod conjugates, respectively.

Regarding maximum temperature (Tₘₐₓ) at 5.1 W/cm², the experimental Tₘₐₓ of the gold nanorods is 25.4° C. whereby the experimental Tₘₐₓ of the gold nanorod conjugates is 48.3° C., the latter being greater by a factor of 1.9 relative to the former.

Regarding Tₘₐₓ at 2.5 W/cm², the experimental Tₘₐₓ of the gold nanorods is 15.8° C. whereby the experimental Tₘₐₓ of the gold nanorod conjugates is 22.1° C., the latter being greater by a factor of 1.4 relative to the former.

Regarding Tₘₐₓ at 1.2 W/cm², the experimental Tₘₐₓ of the gold nanorods is 7.7° C. whereby the experimental Tₘₐₓ of the gold nanorod conjugates is 7.7° C.

Regarding Tₘₐₓ at 0.3 W/cm², the experimental Tₘₐₓ of the gold nanorods is 1.7° C. whereby the experimental Tₘₐₓ of the gold nanorod conjugates is 4.8° C., the latter being greater by a factor of 4.7 relative to the former.

For all power densities (5.1 W/cm², 2.5 W/cm², 1.2 W/cm², and 0.3 W/cm²), theoretical calculations yield the Tₘₐₓ of the gold nanorod conjugates being greater than that of the gold nanorods by a factor of 1.8.

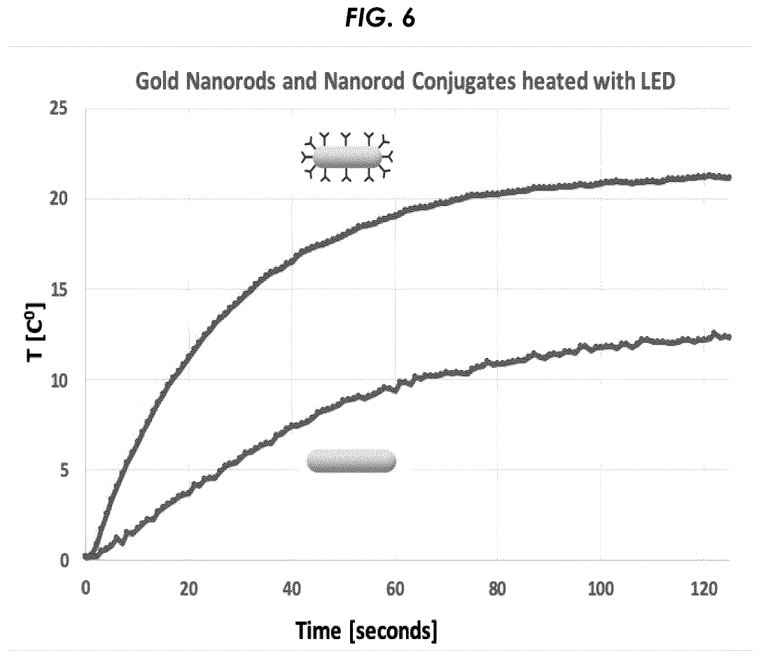

FIG. 6 shows the experimental heating temperature profile of gold nanorods and gold nanorod conjugates with an LED light source, whereby as with the NIR laser, the Tₘₐₓ and the rate of the temperature increase is greater for gold nanorod conjugates as compared to gold nanorods.

Regarding gold nanorods, such nanoparticles may exhibit strong extinction of light in the NIR spectrum caused by longitudinal and transverse surface plasmon resonances. These coherent resonance oscillations of the conduction electrons may result in enhanced extinction of light and heat generation through electron-electron and electron-phonon relaxation processes, whereby such effect can be useful for photothermal therapy.

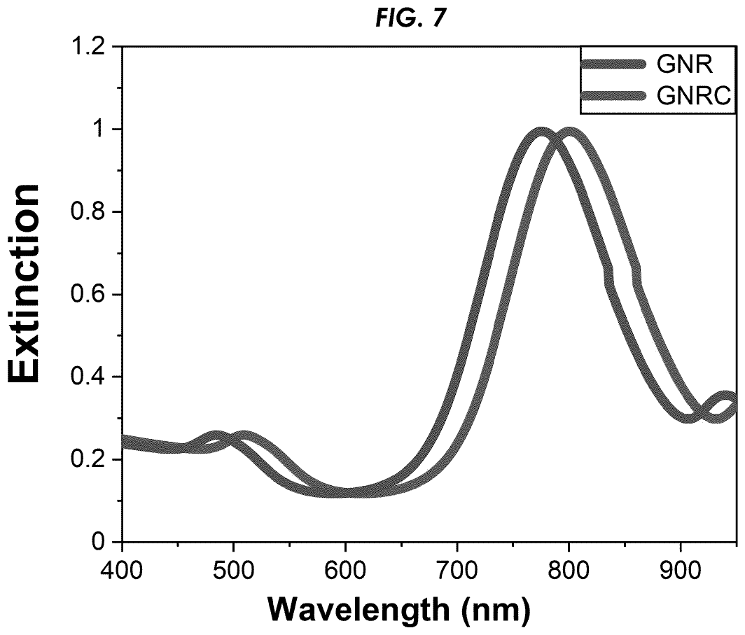

Illustratively, as shown in FIG. 7, the UV-vis extinction spectra of gold nanorods (GNR, blue line) and gold nanorod conjugates with the tumor-targeting NIR dye of Formula II conjugated to the gold nanorods (GNRC, red line) have two peaks corresponding to the longitudinal and transverse surface plasmon resonance modes. The shift in the extinction peaks of the gold nanorod conjugates compared to the gold nanorods is caused by the increase of the local dielectric permittivity of the gold nanorod conjugates.

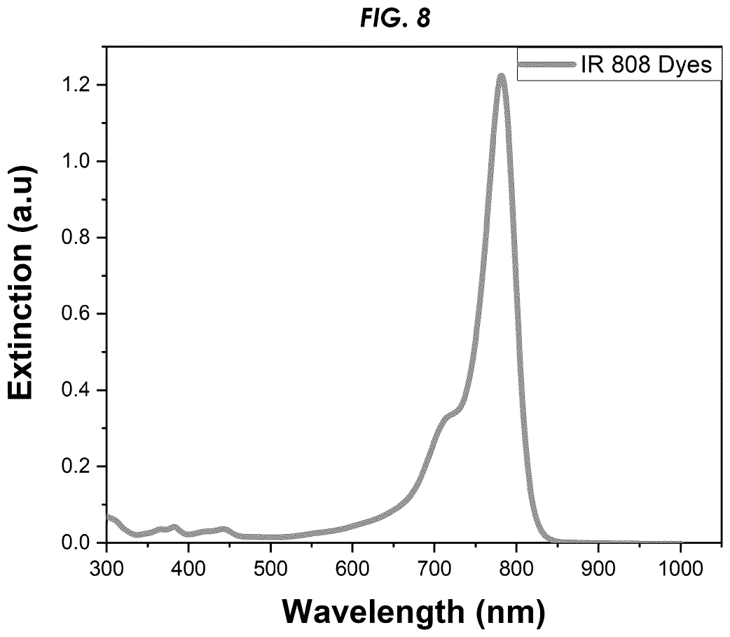

The UV-vis extinction spectra of the tumor-targeting NIR dye of Formula II is shown in FIG. 8.

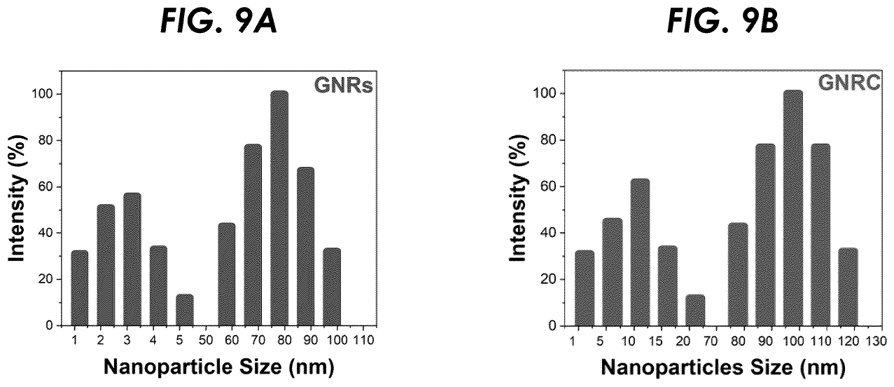

FIGS. 9A and 9B illustrate the hydrodynamic size of gold nanorods and gold nanorod conjugates with the tumor-targeting NIR dye of Formula II conjugated to the gold nanorods, respectively, whereby both show a bimodal distribution with maxima around 3 nm and 80 nm for the gold nanorods (GNRs) and 10 nm and 100 nm for the gold nanorod conjugates (GNRC). The larger size distribution of the gold nanorod conjugates can be due to the decreased aspect ratio resulting from the addition of the tumor-targeting NIR dye to the surface of the gold nanorods.

Now regarding cancer diagnostic agents, the present conjugate including (i) a tumor-targeting NIR dye attached to a gold nanoparticle (and in particular, a gold nanorod) or (ii) a zwitterionic tumor-targeting NIR dye attached to a gold nanoparticle (and in particular, a gold nanorod) or (iii) a tumor-targeting NIR dye attached to a zwitterionic coated gold nanoparticle (and in particular, a gold nanorod) or (iv) a zwitterionic tumor-targeting NIR dye attached to a zwitterionic coated gold nanoparticle (and in particular, a gold nanorod) may be useful as such since the NIR dye can emit NIR light for corresponding detection and imaging/visualization of tumor location.

Regarding the present conjugate including (i) a tumor-targeting NIR dye attached to a gold nanoparticle (and in particular, a gold nanorod) or (ii) a zwitterionic tumor-targeting NIR dye attached to a gold nanoparticle (and in particular, a gold nanorod) or (iii) a tumor-targeting NIR dye attached to a zwitterionic coated gold nanoparticle (and in particular, a gold nanorod) or (iv) a zwitterionic tumor-targeting NIR dye attached to a zwitterionic coated gold nanoparticle (and in particular, a gold nanorod), depending on the distance between the dye molecule and the surface of the nanorod, there may be either radiative or non-radiative decay of the emission from the excited dipole in the molecule. For imaging purposes, the enhancement of the radiative decay rate may be desirable in that it can result in enhanced fluorescence by the dye molecule. In addition to the radiative decay, there also exists a non-radiative decay channel of the excited dye molecule which may be useful for hyperthermia applications, as the non-radiative decay of the dye molecules may allow achievement of a desired temperature elevation with a lesser concentration of nanoconjugates and/or lesser irradiation.

It is herein contemplated that silver nanorods may perform the same as or similar to gold nanorods for use with the present invention.

Now again regarding cancer therapeutic agents, as another illustrative example, a hyperthermia agent can comprise a magnetic nanoparticle, which may include materials based on iron oxide nanoparticles (IONPs), such as Fe₃O₄, or materials such as FeCo, FePt, or Fe₁₋ₓSiₓ, which can heat in an alternating magnetic field.

As to particular embodiments, a conjugate including a tumor-targeting NIR dye and an iron oxide nanoparticle which may be useful with the present invention can have Formula XVI as follows:

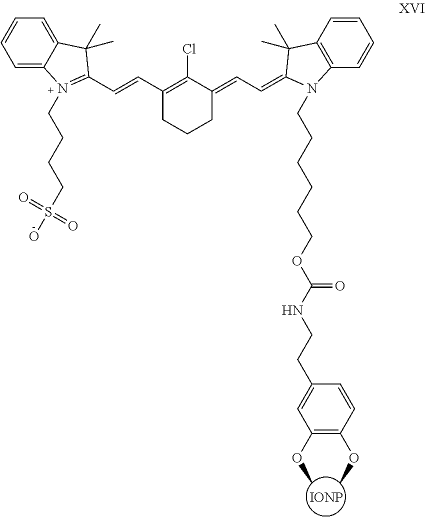

Of note, a tumor-targeting NIR dye having a catechol functionality can directly conjugate to an iron oxide nanoparticle via a relatively strong catechol-IONP bond (such as the conjugate shown in Formula XVI).

As stated above, a conjugate comprising an iron oxide nanoparticle can heat in an alternating magnetic field and function as a hyperthermia agent.

Following, as to particular embodiments, a method of using the present conjugate including (i) a tumor-targeting NIR dye attached to an iron oxide nanoparticle or (ii) a zwitterionic tumor-targeting NIR dye attached to an iron oxide nanoparticle or (iii) a tumor-targeting NIR dye attached to a zwitterionic coated iron oxide nanoparticle or (iv) a zwitterionic tumor-targeting NIR dye attached to a zwitterionic coated iron oxide nanoparticle can be to administer the conjugate to a patient (such as via injection), whereby following administration, the NIR dye can direct the conjugate to the target tumor cell population. Subsequently, an alternating magnetic field can be generated, whereby exposure to the alternating magnetic field can result in heating of the iron oxide nanoparticles and consequent induction of localized hyperthermia in the tumor cells.

As to other particular embodiments, a method of using the present conjugate including (i) a tumor-targeting NIR dye attached to an iron oxide nanoparticle or (ii) a zwitterionic tumor-targeting NIR dye attached to an iron oxide nanoparticle or (iii) a tumor-targeting NIR dye attached to a zwitterionic coated iron oxide nanoparticle or (iv) a zwitterionic tumor-targeting NIR dye attached to a zwitterionic coated iron oxide nanoparticle can be to administer the conjugate to a patient (such as via injection), whereby following administration, the NIR dye can direct the conjugate to the target tumor cell population. Subsequently, an NIR field can be generated (for example via laser irradiation or light-emitting diode (LED) irradiation), whereby upon exposure to the NIR field, the iron oxide nanoparticles can convert absorbed light into heat and induce localized hyperthermia in the tumor cells, with minimal damage to intervening and surrounding normal tissue.

Further, the present conjugate including (i) a tumor-targeting NIR dye attached to an iron oxide nanoparticle or (ii) a zwitterionic tumor-targeting NIR dye attached to an iron oxide nanoparticle or (iii) a tumor-targeting NIR dye attached to a zwitterionic coated iron oxide nanoparticle or (iv) a zwitterionic tumor-targeting NIR dye attached to a zwitterionic coated iron oxide nanoparticle may be useful as a diagnostic agent, as (i) the NIR dye can emit NIR light and/or (ii) the iron oxide nanoparticles can function as a magnetic resonance imaging (MRI) contrast agent for corresponding detection and imaging/visualization of tumor location.

As to particular embodiments, carbon nanoparticles, such as carbon nanospheres, carbon nanoshells, carbon nanotubes, other so-called “lossy dielectric” materials, or the like, can also function as strong NIR light absorbers and accordingly, may be useful with the present invention. Of note, carbon nanotubes can be targeted to specific cells either through direct covalent functionalization to a targeting moiety (such as a tumor-targeting NIR dye), or through non-covalent wrapping of a targeting moiety (such as a tumor-targeting NIR dye).

Now regarding nanoparticles (such as gold nanoparticles or iron oxide nanoparticles), an obstacle to their medical application and translation to the clinic, for example as hyperthermia agents, may be the tendency of such particles to accumulate in the liver and spleen as a result of opsonization and scavenging by the mononuclear phagocyte system. Specifically, nanoparticles may be highly prone to association with proteins, lipids, and other biomolecules, leading to formation of a dynamic “biomolecular corona” that can make it difficult to predict and/or control their behavior in vivo. Binding of serum proteins followed by recognition and engulfing by phagocytic cells can be a particularly common fate of many nanoparticles, resulting in rapid clearance from the bloodstream. Zwitterionic coatings may counter this issue and render nanoparticles more biocompatible, for example by reducing the rate and/or extent of non-specific adsorption of proteins and lipids to the nanoparticle surface, thereby inhibiting production of the biomolecular corona. As described above, zwitterions may obtain their antifouling properties via strong electrostatic interactions of their positively and negatively charged groups with polar water molecules, creating a dense and stable hydration shell which can provide a physical and energetic barrier that can prevent undesired adsorption. In addition, as the zwitterionic coating can be overall neutrally charged, the formation of an ion pair with a charged adsorbent may be less likely.

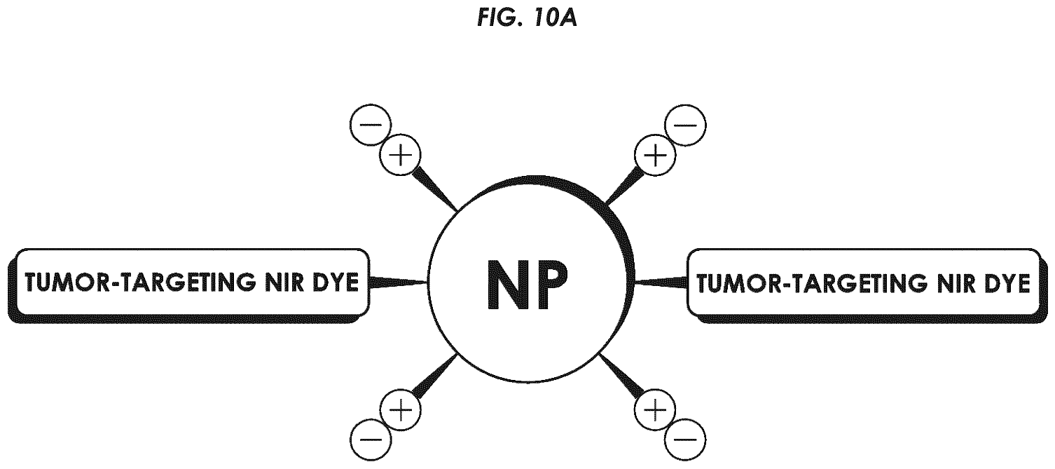

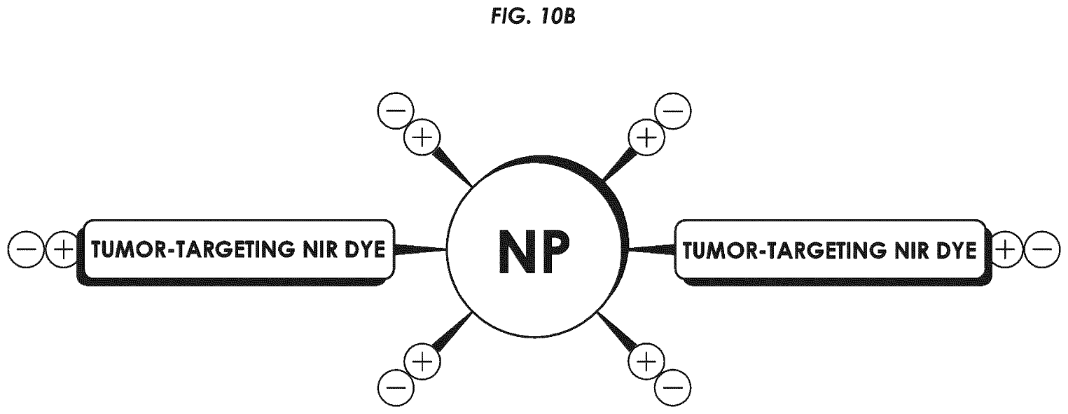

FIG. 10A illustrates a particular embodiment of the present conjugate which includes a tumor-targeting NIR dye attached to a zwitterionic nanoparticle.

FIG. 10B illustrates a particular embodiment of the present conjugate which includes a zwitterionic tumor-targeting NIR dye attached to a zwitterionic nanoparticle.

As to particular embodiments, illustrative examples of zwitterions which may be useful with the present invention when attached to a nanoparticle include sulfobetaine, carboxybetaine, phosphorylcholine, and those shown in FIG. 2.

As to particular embodiments, regarding attachment, a 5:1 ratio of zwitterion: tumor-targeting NIR dye or zwitterion: zwitterionic tumor-targeting NIR dye may be useful with the present invention.

As to particular embodiments, a conjugate including a tumor-targeting NIR dye attached to a zwitterionic nanoparticle (such as a gold nanoparticle, for example a gold nanorod) can have Formula XVII as follows:

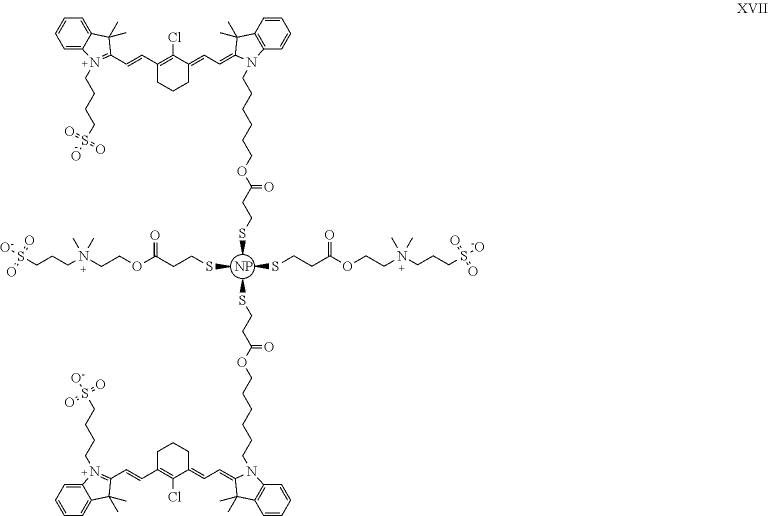

As to particular embodiments, a conjugate including a tumor-targeting NTR dye attached to a zwitterionic nanoparticle (such as a gold nanoparticle, for example a gold nanorod) can have Formula XVIII as follows:

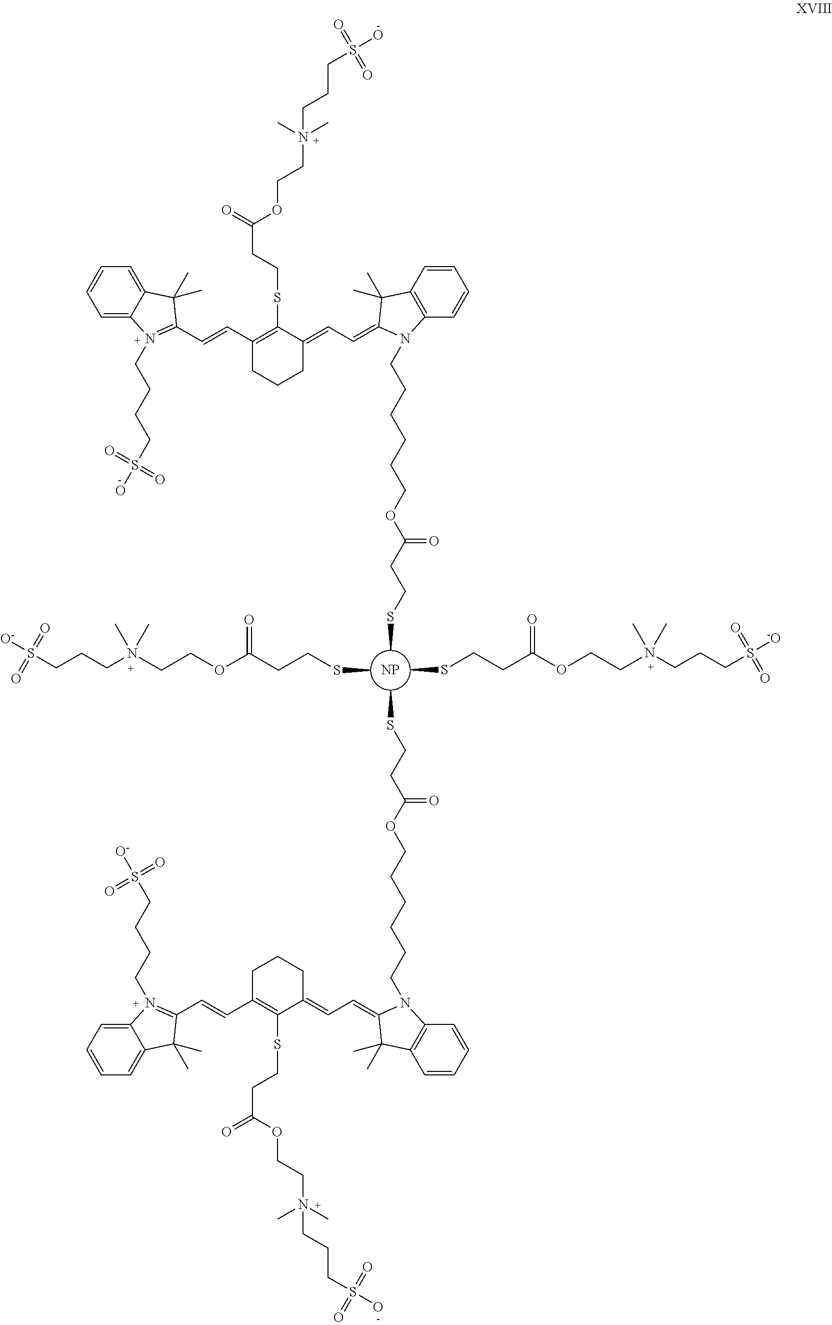

As to particular embodiments, a conjugate including a tumor-targeting NIR dye attached to a zwitterionic nanoparticle (such as a gold nanoparticle, for example a gold nanorod) can have Formula XIX as follows:

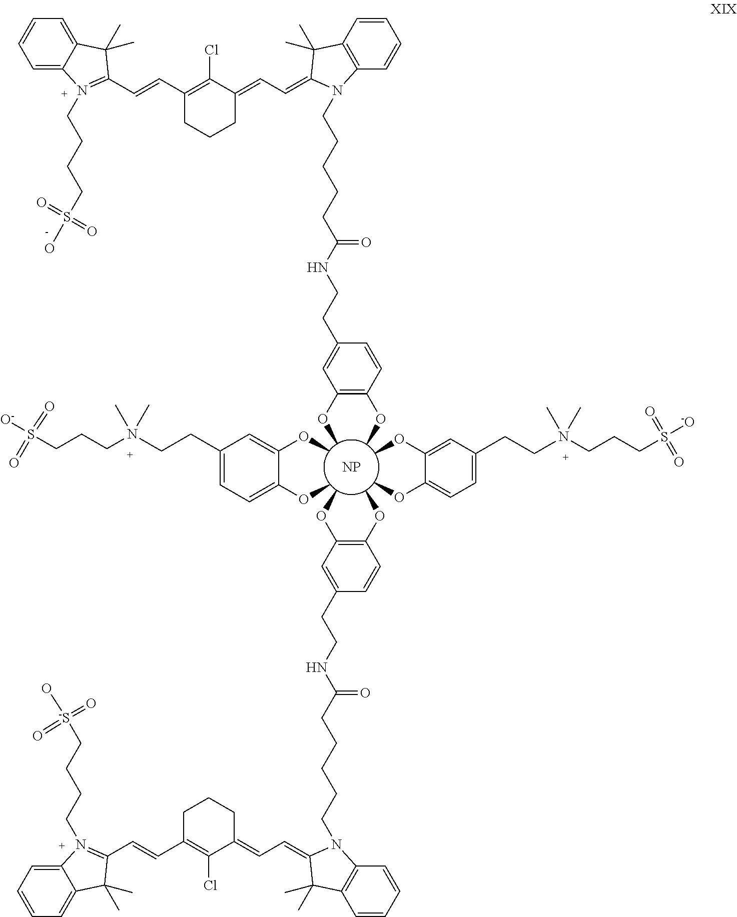

As to particular embodiments, a conjugate including a tumor-targeting NTR dye attached to a zwitterionic nanoparticle (such as a gold nanoparticle, for example a gold nanorod) can have Formula XX as follows:

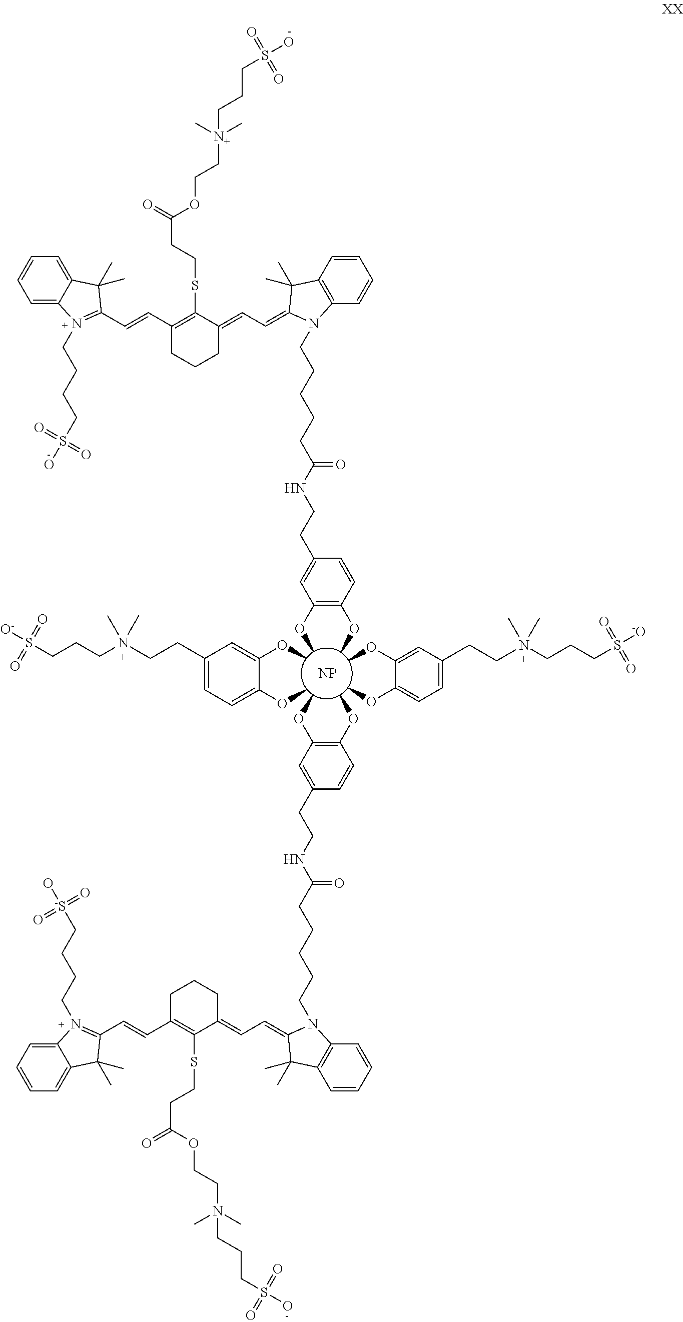

Now again regarding therapeutic agents, as to particular embodiments, a tumor-targeting NIR dye or a zwitterionic tumor-targeting NIR dye can be coupled to (for example via conjugation) a chemotherapeutic agent via various linkers for use in chemotherapy, as per the present invention. For example, a few conventional chemotherapeutics which may be conjugated to a tumor-targeting NIR dye, as per the present invention, can include actinomycin, all-trans retinoic acid, azacitidine, azathioprine, bleomycin, bortezomib, carboplatin, capecitabine, cisplatin, chlorambucil, cyclophosphamide, cytarabine, daunorubicin, doxifluridine, doxorubicin, epirubicin, epothilone, etoposide, fluorouracil, gemcitabine, hydroxyurea, idarubicin, imatinib, irinotecan, mechlorethamine, methotrexate, mitoxantrone, nitrogen mustard, oxaliplatin, paclitaxel, pemetrexed, teniposide, tioguanine, topotecan, valrubicin, vemurafenib, vinblastine, vincristine, vindesine, or the like.

For use with the present invention, the chemotherapeutic agent can be fully pharmacologically active following conjugation or alternatively, the chemotherapeutic agent can be a prodrug, meaning an inactive or less active derivative of the chemotherapeutic agent which can be transformed into its pharmacologically active form via metabolism, such as by a chemical and/or enzymatic mechanism, whereby the pharmacologically active form can be effective to kill tumor cells.

In contrast to administration in its pharmacologically active form, administering a chemotherapeutic agent in its prodrug form can improve the absorption, distribution, metabolism, and/or excretion (ADME) of said chemotherapeutic agent. For example, a prodrug form may have improved bioavailability relative to its pharmacologically active form, which may be beneficial for chemotherapeutic agents that are poorly absorbed via the gastrointestinal tract. Additionally, the prodrug form may facilitate how selectively the chemotherapeutic agent interacts with cells or processes that are not its intended target, which may reduce unintended, undesirable, and/or adverse side effects of the chemotherapeutic agent.

As many hyperthermia agents, such as gold nanoparticles, iron oxide nanoparticles, or the like, can be inherently nontoxic to cells, a prodrug form of these hyperthermia agents to form the present conjugate may not be as necessary as a prodrug form of a chemotherapeutic agent for the present conjugate. For example, doxorubicin (DOX), a commonly used clinical anticancer agent, can be non-specific and thus, DOX can be toxic to normal cells, especially those of the heart, liver, and kidneys. Following, it is herein contemplated that the adverse side effects of DOX may be reduced, minimized, or eliminated by incorporation into the present conjugate which targets DOX via the present NIR dye or zwitterionic NTR dye to the tumor site, whereby the specific linker which conjugates DOX to the dye may contribute to said decrease in the adverse side effects of DOX, as the linker can affect the pharmacokinetics, pharmacodynamics, stability, toxicity, etc. of the chemotherapeutic agent incorporated into the conjugate. A preferred linker may ensure adequate stability of the prodrug form of the chemotherapeutic agent in the circulation, effectively prevent premature release of the chemotherapeutic agent proximate an unintended target, and timely facilitate the release and/or pharmacological activation of the chemotherapeutic agent only proximate the targeted tumor cells.

Linkers which may be useful to conjugate the present tumor-targeting NIR dye and a therapeutic agent, such as a chemotherapeutic agent, can be classified as cleavable and non-cleavable linkers. Non-cleavable linkers can change the activity of the active metabolite, which may impact efficacy, toxicity, and trafficking, thereby presenting significant limitations. Following, cleavable linkers may be more desirable for use with the present conjugates and can significantly contribute to the success thereof in effectively killing the target tumor cell population.

In particular, cleavable linkers which are stable in the circulation for a long period of time and use the inherent properties of tumor cells to selectively release the chemotherapeutic agent from the conjugate may be especially desirable.

As to particular embodiments, a cleavable linker with glutathione (GSH) sensitivity (redox promoted) may be used with the present conjugate to link a tumor-targeting NIR dye and a chemotherapeutic agent. Certain cancer cells have higher intracellular GSH concentrations than in plasma, whereby the elevated GSH levels may reduce disulfide bonds to maintain intracellular redox balance. This disulfide bond may be employed as cleavable linker which can remain stable in the blood system and release active payloads exclusively in tumor cells with elevated GSH levels. Illustrative examples of two cleavable linkers with GSH sensitivity are shown in Formulas XXI (faster release/less stability) and XXII (lower release/better stability).

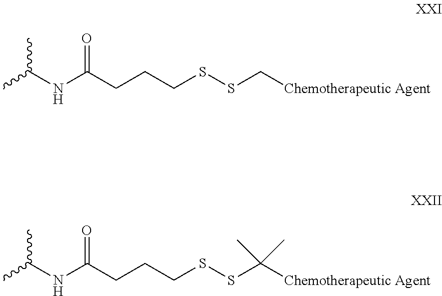

As to other particular embodiments, a cleavable linker with pH sensitivity (acid induced) may be used with the present conjugate to link a tumor-targeting NIR dye and a chemotherapeutic agent. Acid-sensitive cleavable linkers can take advantage of the lower pH of endosomes (pH=5-6) and lysosomes (pH=4.8) relative to the cytoplasm (pH=7.4) to trigger the hydrolysis of an acid-labile group(s) within the linker. The three bond types shown in Formulas XXIII (hydrazone-based bond), XXIV (carbonate-based bond), and XXV (silyl ether-based bond) can be used with cleavable linkers having pH sensitivity:

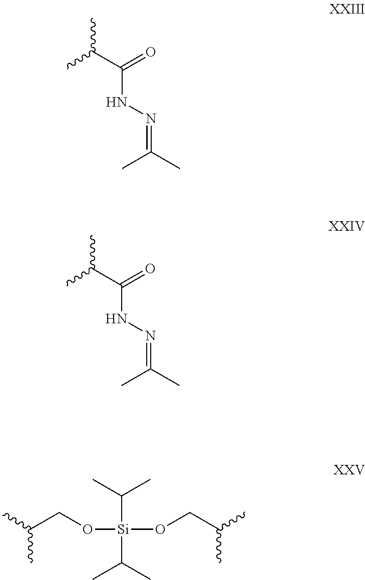

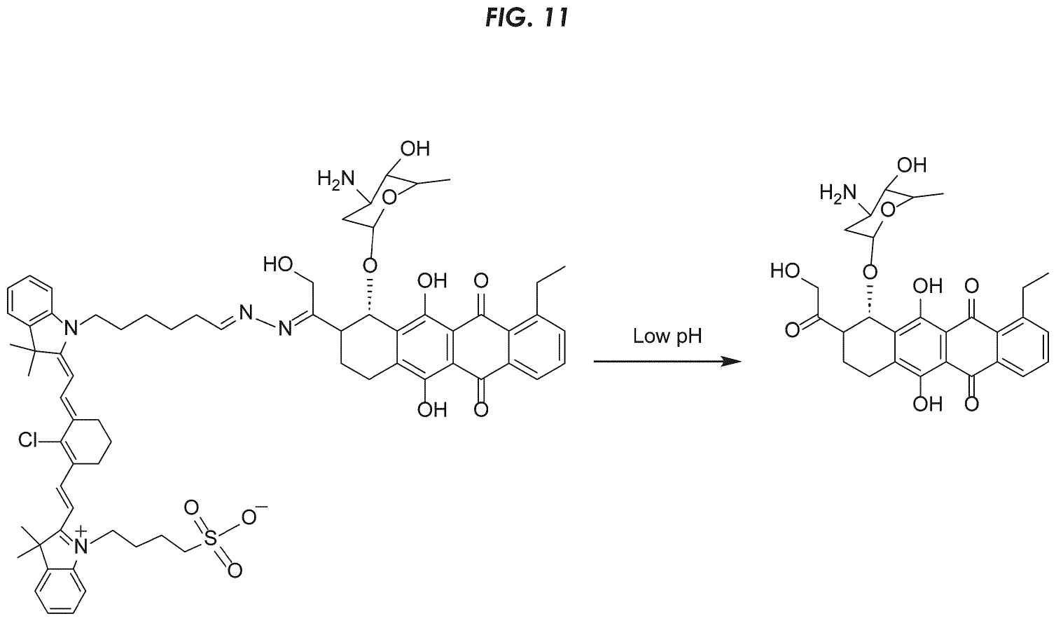

As but one illustrative example, FIG. 11 shows a particular embodiment of the present conjugate including DOX conjugated to a tumor-targeting NIR dye via a cleavable linker with pH sensitivity having an acid-labile hydrazone bond which can be cleaved once the conjugate enters the more acidic environment of a tumor site/cell, thus releasing DOX at the targeted location.

As to other particular embodiments, a cleavable linker with protease sensitivity (enzyme catalyzed) may be used with the present conjugate to link a tumor-targeting NIR dye and a chemotherapeutic agent. Protease-sensitive cleavable linkers can employ proteases found in the lysosomes of tumor cells which recognize and cleave specific peptide sequences in the linker. Dubowchik and Firestone et al. pioneered the discovery of the valine-citrulline (VC) dipeptide (as shown in Formula XXVI below) as an intracellular cleavage mechanism by cathepsin B which can be overexpressed in tumor cells, thus allowing the linked chemotherapeutic agent to be released precisely proximate the tumor site. Likewise, some tumor cells overexpress MMP-2 proteases which can recognize and cleave specific peptide sequences in the linker, such as a synthetic octapeptide (GPLGIAGQ) (as shown in Formula XXVII below). Further, cleavable linkers with protease sensitivity may be stable in the systemic circulation due to the presence of protease inhibitors in the blood.

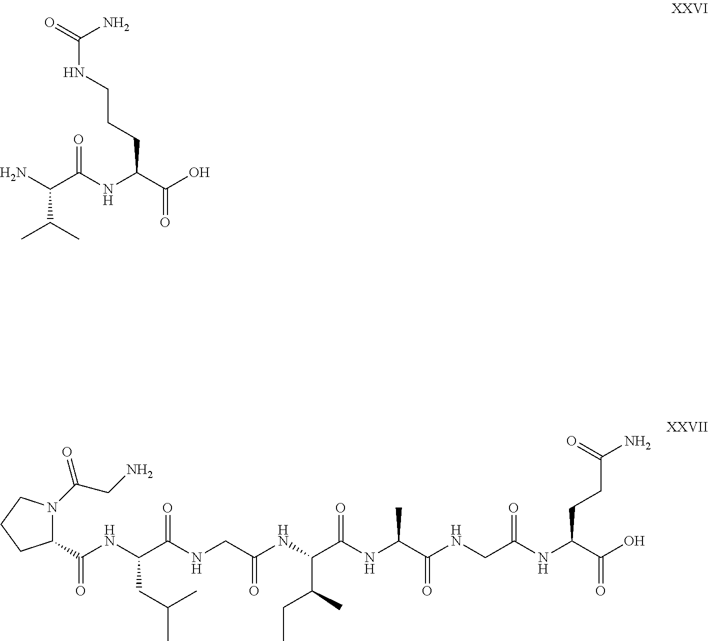 [SMILES: CC(C)[C@H](N)C(=O)N[C@@H](CCCNC(N)=O)C(=O)O ; CC[C@H](C)[C@H](NC(=O)CNC(=O)[C@H](CC(C)C)NC(=O)[C@@H]1CCCN1C(=O)CN)C(=O)N[C@@H](C)C(=O)NCC(=O)N[C@@H](CCC(N)=O)C(=O)O]

Protease-sensitive cleavable peptide linkers have been developed as a critical component of antibody drug conjugates (ADC), whereby the linker can play a vital role in the overall success of the ADC drug as a result of their superior plasma stability and controlled payload release mechanism; nine of the fourteen approved ADC drugs use protease-sensitive cleavable peptide linkers. For example, brentuximab vedotin (ADCETRIS®) uses a valine-citrulline linker.

Instead of using an antibody as a tumor-targeting agent, particular embodiments of the present conjugate employ (i) tumor-targeting NIR dyes to selectively and effectively deliver a chemotherapeutic prodrug to targeted tumor cells, and (ii) protease-sensitive cleavable peptide linkers to release and/or pharmacologically activate the chemotherapeutic agent only proximate the targeted tumor site. Formulas XXVIII, XXIX, and XXX show exemplary tumor-targeting NIR dyes conjugated to various chemotherapeutic agents via protease-sensitive cleavable peptide linkers.

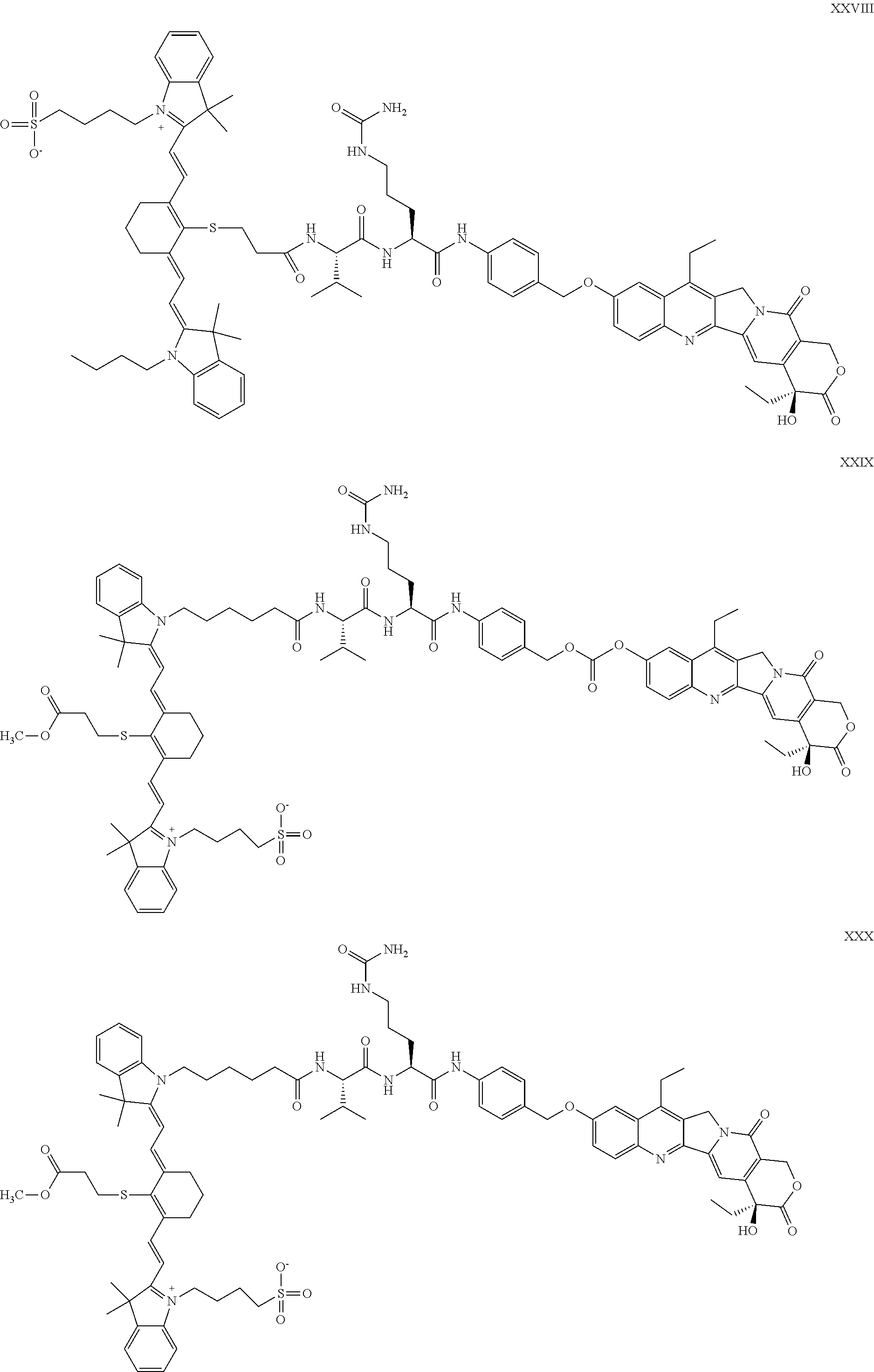

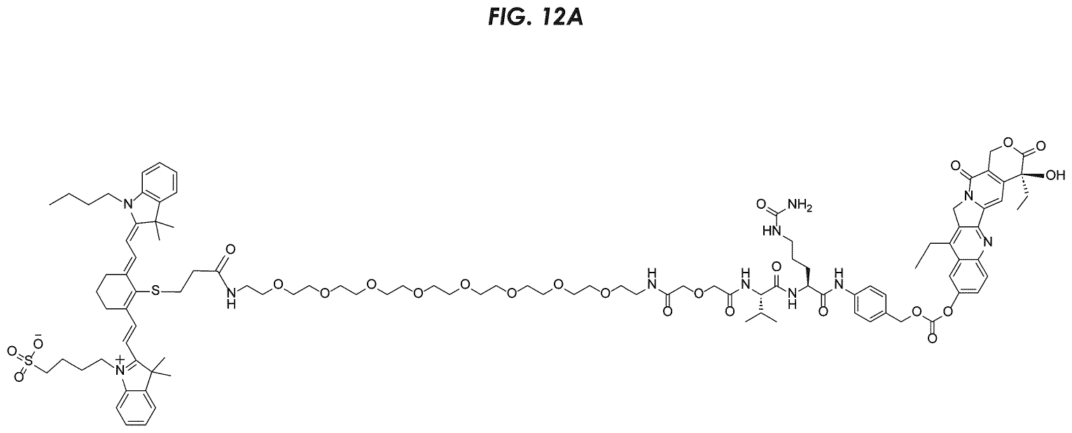

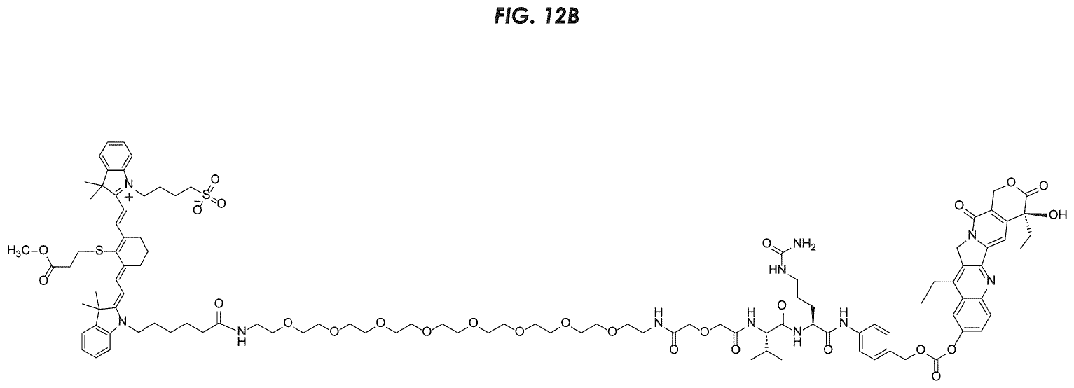

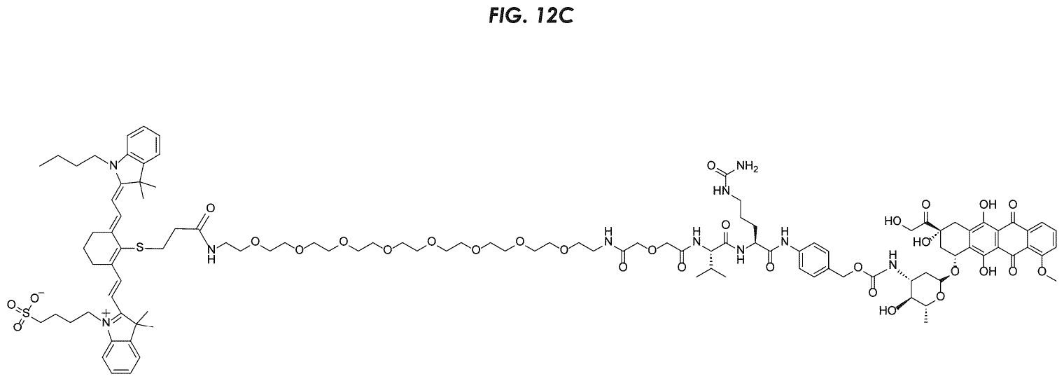

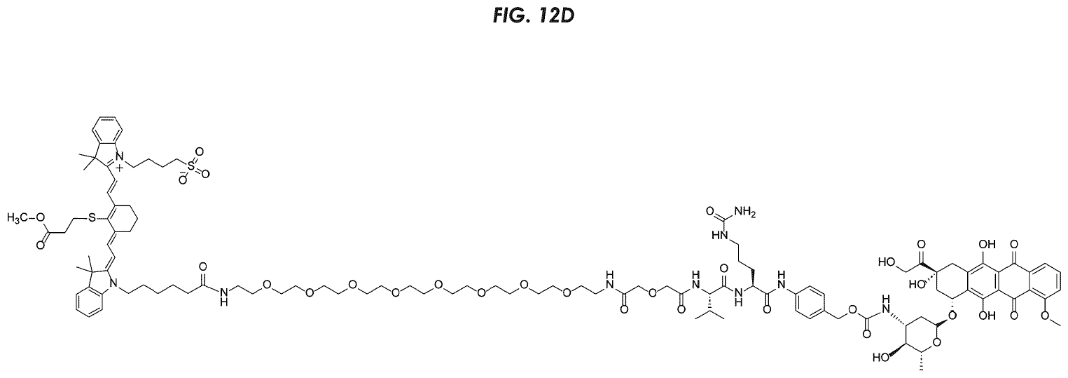

As to particular embodiments, the present linker can include polyethylene glycol (PEG) (as shown in FIGS. 12A and 12B which include SN-38 as the chemotherapeutic agent, and FIGS. 12C and 12D which include DOX as the chemotherapeutic agent), whereby the PEG may increase solubility and provide the desired drug metabolism and pharmacokinetics (DMPK) of the delivered chemotherapeutic agent.

As to particular embodiments, the present conjugate can include two linkers, the first linker linking the tumor-targeting NIR dye and the protease-sensitive cleavable peptide, and the second linker linking the protease-sensitive cleavable peptide and the chemotherapeutic agent. The first linker must be stable (for example in the circulation) but need not necessarily be cleavable, whereas the second linker must be stable (for example in the circulation) and cleavable once the conjugate is delivered to the tumor site such that the chemotherapeutic agent can be released and/or pharmacologically activated proximate the targeted tumor site. Correspondingly, a self-immolative spacer can be inserted between the protease-sensitive cleavable peptide and the chemotherapeutic agent to facilitate effective release of the latter.

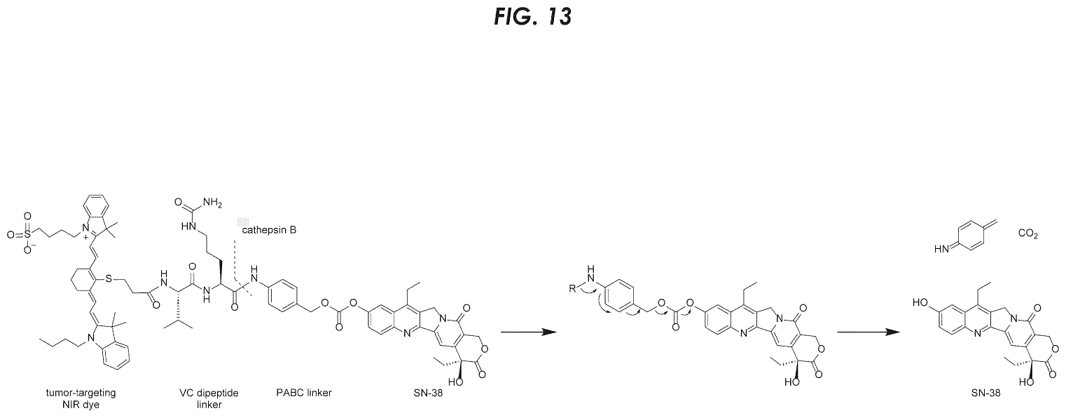

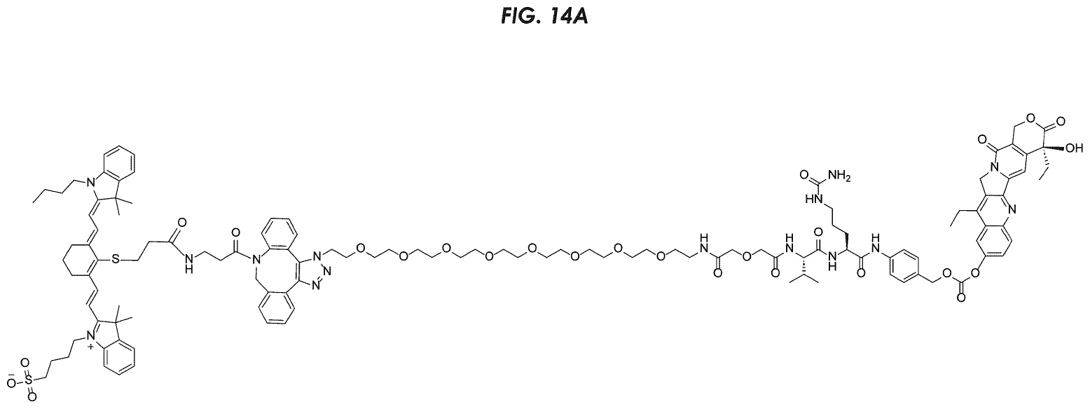

As an illustrative example, cathepsin B can cleave a linker including the VC dipeptide to release the conjugate portion including the chemotherapeutic agent, such as SN-38, and a self-immolative para-aminobenzyl carbamate (PABC) spacer. Subsequently, PABC can undergo spontaneous 1,6-elimination in an acidic environment to release carbon dioxide, para azaquinone methide, and SN-38, as shown in FIG. 13.

In addition to the linker including the VC dipeptide, other exemplary linkers having peptide sequences with protease sensitivity which may be useful with the present conjugate include those shown in Formulas XXXI (valine-alanine), XXXII (valine-glycine), XXXIII (glycine-glycine), XXXIV (alanine-alanine-asparagine), and XXXV (phenylalanine-lysine).

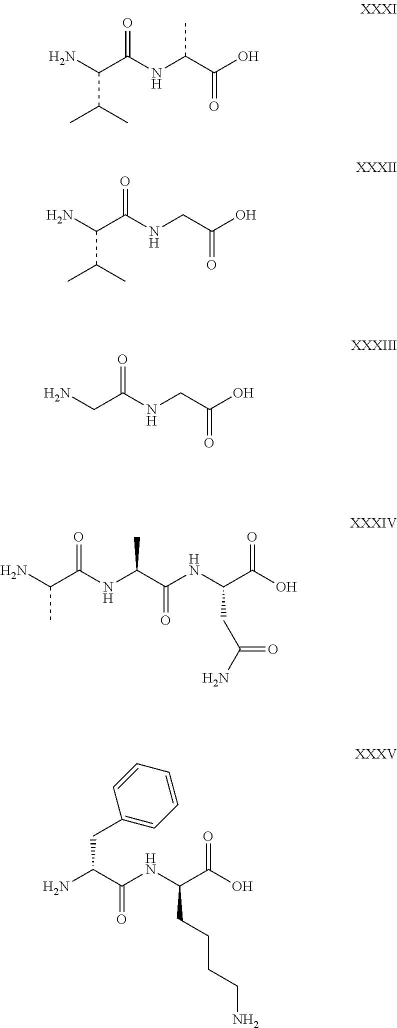 [SMILES: CC(C)[C@H](N)C(=O)N[C@H](C)C(=O)O ; CC(C)[C@H](N)C(=O)NCC(=O)O ; NCC(=O)NCC(=O)O ; C[C@H](N)C(=O)N[C@@H](C)C(=O)N[C@@H](CC(N)=O)C(=O)O ; NCCCC[C@@H](NC(=O)[C@H](N)Cc1ccccc1)C(=O)O]

Formulas XXXVI through XXXXI show exemplary conjugates of the present invention which include a tumor-targeting NIR dye having a zwitterion functional group(s), a chemotherapeutic agent prodrug, a cathepsin B cleavable linker, and a self-immolative spacer which facilitates effective release of the chemotherapeutic agent prodrug. Due to the zwitterionic functionality of the tumor-targeting NIR dye and the resultant high hydration, PEG may be omitted from these conjugates.

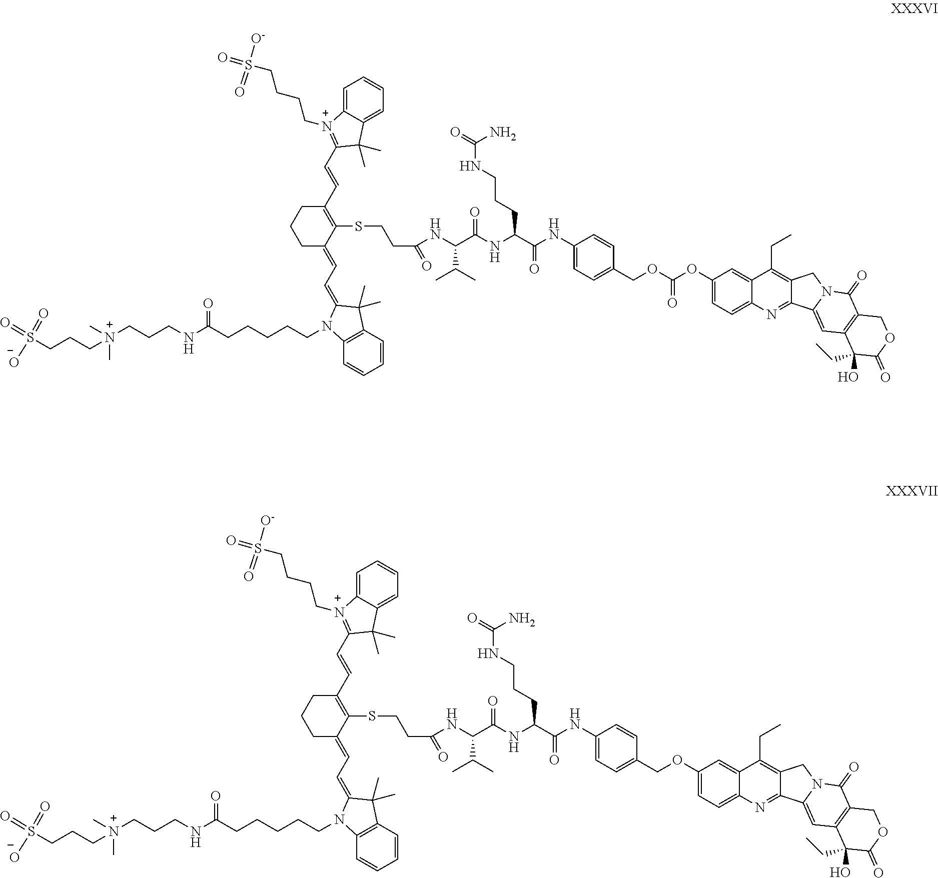 [SMILES: CCc1c2c(nc3ccc(OC(=O)OCc4ccc(NC(=O)[C@H](CCCNC(N)=O)NC(=O)[C@@H](NC(=O)CCSC5=C(/C=C/C6=[N+](CCCCS(=O)(=O)[O-])c7ccccc7C6(C)C)CCC/C5=C\C=C5\N(CCCCCC(=O)NCCC[N+](C)(C)CCCS(=O)(=O)[O-])c6ccccc6C5(C)C)C(C)C)cc4)cc13)-c1cc3c(c(=O)n1C2)COC(=O)[C@]3(O)CC ; CCc1c2c(nc3ccc(OCc4ccc(NC(=O)[C@H](CCCNC(N)=O)NC(=O)[C@@H](NC(=O)CCSC5=C(/C=C/C6=[N+](CCCCS(=O)(=O)[O-])c7ccccc7C6(C)C)CCC/C5=C\C=C5\N(CCCCCC(=O)NCCC[N+](C)(C)CCCS(=O)(=O)[O-])c6ccccc6C5(C)C)C(C)C)cc4)cc13)-c1cc3c(c(=O)n1C2)COC(=O)[C@]3(O)CC]

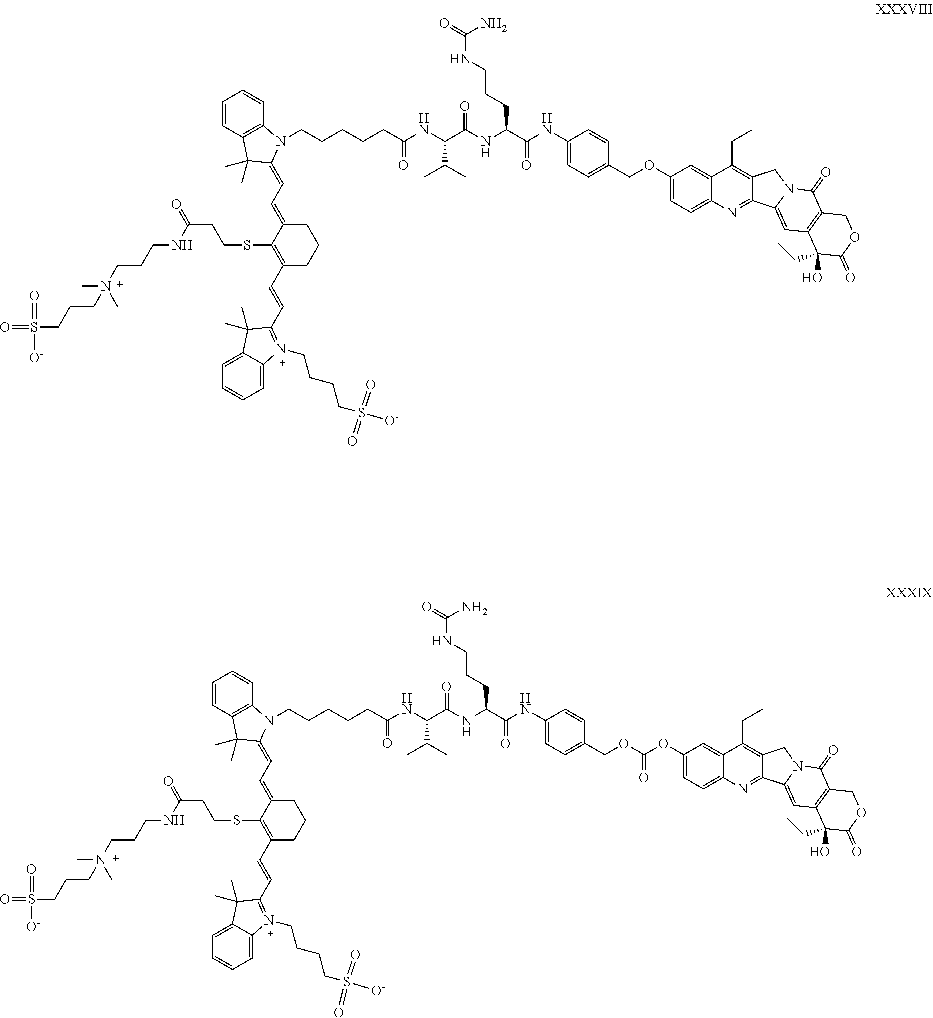 [SMILES: CCc1c2c(nc3ccc(OCc4ccc(NC(=O)[C@H](CCCNC(N)=O)NC(=O)[C@@H](NC(=O)CCCCCN5/C(=C/C=C6\CCCC(/C=C/C7=[N+](CCCCS(=O)(=O)[O-])c8ccccc8C7(C)C)=C6SCCC(=O)NCCC[N+](C)(C)CCCS(=O)(=O)[O-])C(C)(C)c6ccccc65)C(C)C)cc4)cc13)-c1cc3c(c(=O)n1C2)COC(=O)[C@]3(O)CC ; CCc1c2c(nc3ccc(OC(=O)OCc4ccc(NC(=O)[C@H](CCCNC(N)=O)NC(=O)[C@@H](NC(=O)CCCCCN5/C(=C/C=C6\CCCC(/C=C/C7=[N+](CCCCS(=O)(=O)[O-])c8ccccc8C7(C)C)=C6SCCC(=O)NCCC[N+](C)(C)CCCS(=O)(=O)[O-])C(C)(C)c6ccccc65)C(C)C)cc4)cc13)-c1cc3c(c(=O)n1C2)COC(=O)[C@]3(O)CC]

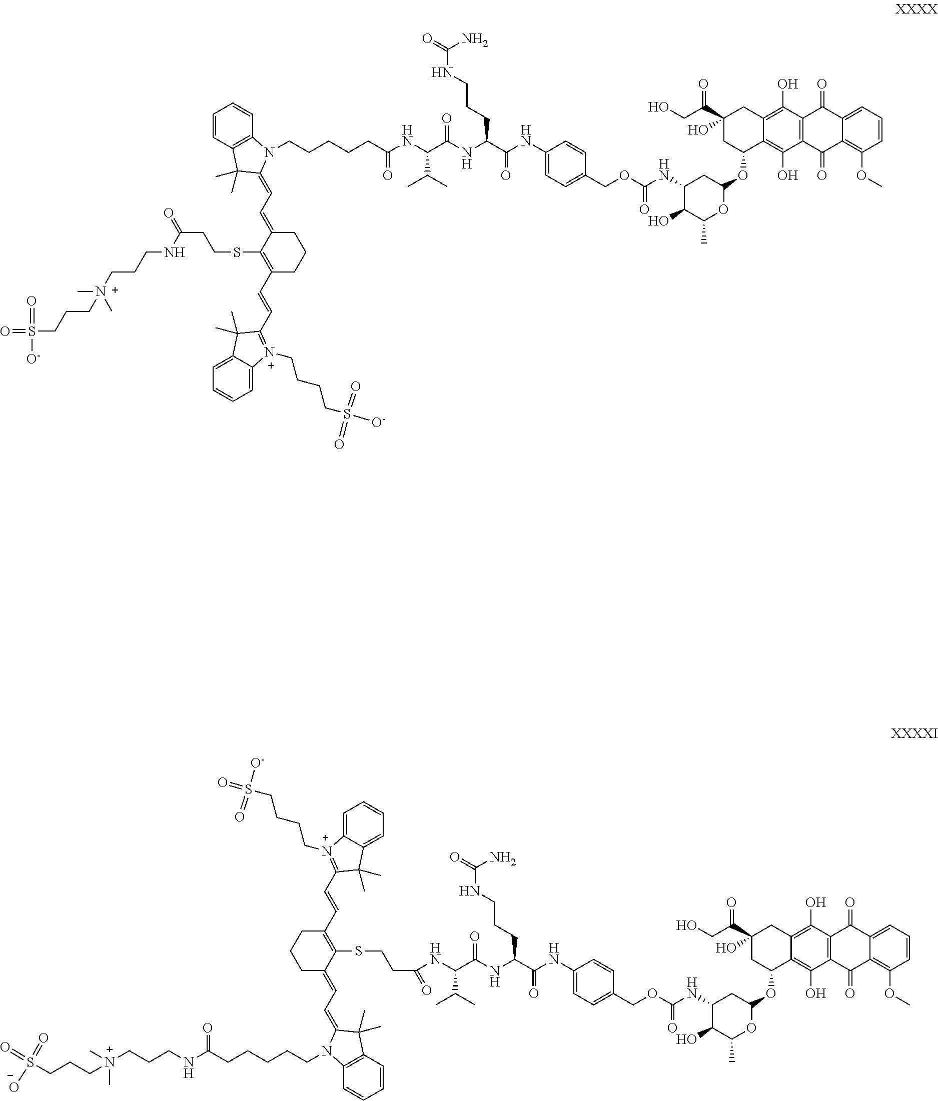

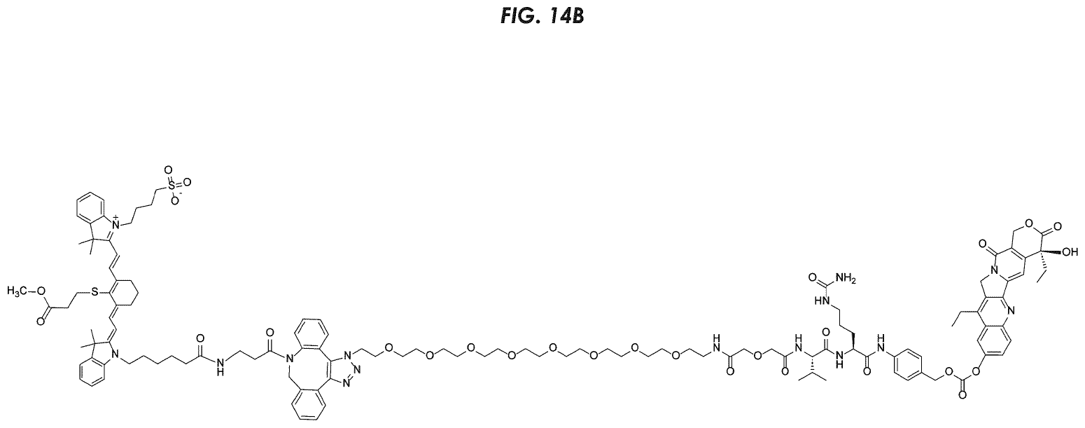

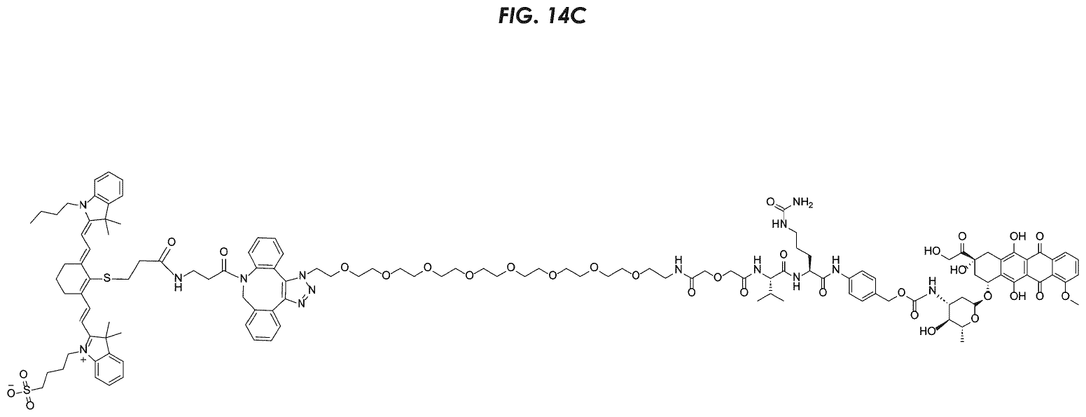

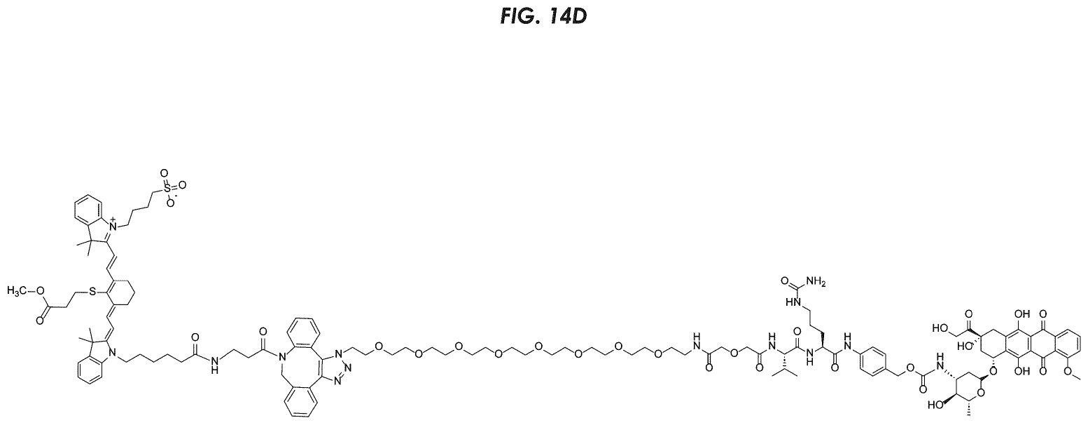

As to particular embodiments, click chemistry can be used to link the tumor-targeting NIR dye and the cleavable peptide sequence. As but one illustrative example, dibenzocyclooctyne (DBCO) reagents include a highly reactive DBCO group which can react with azide-tagged molecules or biomolecules via copper-free click chemistry. DBCO click chemistry can be run in an aqueous buffer or in organic solvents depending on the property of the substrate molecules. FIGS. 14A and 14B (which include SN-38 as the chemotherapeutic agent) and Formulas FIGS. 14C and 14D (which include DOX as the chemotherapeutic agent) show exemplary conjugates of the present invention made using click chemistry.

As to particular embodiments, a tumor-targeting NIR dye or a zwitterionic tumor-targeting NIR dye can be coupled to (for example via conjugation) a nitroxide radical, as per the present invention. As cancer cells may produce higher levels of free radicals than normal cells due to their active metabolism associated with the dysregulation of various cellular events, these cells can be under constant oxidative stress. While an overproduction of high levels of free radicals can result in detrimental cell damage and ultimately cell death, moderate levels may facilitate cancer cell survival and promote tumor growth. Following, cancer cells may rely heavily on antioxidant enzymes and other adaptive antioxidant defenses to maintain intracellular levels of reactive oxidative species (ROS) within a tolerable threshold and thus protect these cells from damage.

Correspondingly, nitroxide radicals may be suitable cancer therapeutics, as these compounds, either alone or when combined with another therapy (such as a chemotherapeutic), can intensify the oxidative stress in cancer cells, which can lead to cell death.

As to particular embodiments, a tumor-targeting NIR dye or a zwitterionic tumor-targeting NIR dye can be coupled to (for example via conjugation) a photodynamic therapy (PDT) agent, as per the present invention. Generally, PDT includes administration of a photosensitizer, for example via injection into the bloodstream or application to an affected area of skin. Upon activation of the photosensitizer by light, one or more processes may be initiated which can result in tumor cell death. However, accumulation of conventional photosensitizers in tumor cells can be low, whereby an example of one such PDT agent can be chlorin-e6 PSs. It is herein contemplated that the present invention can increase accumulation of PDT agents in tumor cells.

As can be easily understood from the foregoing, the basic concepts of the present invention may be embodied in a variety of ways. The invention involves numerous and varied embodiments of tumor-targeting NIR dyes and associated conjugates and methods for making and using such dyes and associated conjugates.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “therapeutic” should be understood to encompass disclosure of the act of “providing therapy”—whether explicitly discussed or not—and, conversely, were there effectively disclosure of the act of “providing therapy”, such a disclosure should be understood to encompass disclosure of an “therapeutic” and even a “means for providing therapy.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the NIR dyes and associated conjugates herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A composition comprising:
a conjugate comprising:
a tumor-targeting near-infrared dye; and
a therapeutic agent;
wherein said near-infrared dye targets said therapeutic agent to tumor cells.

2. The composition of claim 1, wherein said near-infrared dye comprises a zwitterion.

3. The composition of claim 2, wherein said zwitterion comprises both positively and negatively charged groups.

4. The composition of claim 3, wherein said zwitterion comprises an overall neutral charge.

5. The composition of claim 3, wherein said zwitterion comprises a high hydration capacity.

6. The composition of claim 5, wherein said zwitterion is capable of being highly hydrated via electrostatic interactions of said positively and negatively charged groups with polar water molecules.

7. The composition of claim 6, wherein said interactions result in the formation of a hydration shell.

8. The composition of claim 7, wherein said hydration shell provides a physical and energetic barrier which prevents undesired adsorption.

9. The composition of claim 7, wherein said hydration shell provides a physical and energetic barrier which prevents undesired protein adsorption.

10-18. (canceled)

19. The composition of claim 8, wherein said zwitterion comprises a molecule shown in FIG. 2.

20. The composition of claim 8, wherein said near-infrared dye comprises a cyanine dye.

21. The composition of claim 20, wherein said cyanine dye comprises a heptamethine cyanine dye.

22. The composition of claim 21, wherein said heptamethine cyanine dye has Formula I.

23-31. (canceled)

32. The composition of claim 1 or claim 8, wherein said therapeutic agent comprises a hyperthermia agent.

33. The composition of claim 8, wherein said therapeutic agent comprises a photothermal agent.

34. The composition of claim 32, wherein said hyperthermia agent comprises a metal or semiconducting nanoparticle which is heatable in an electromagnetic field.

35-47. (canceled)

48. The composition of claim 1, wherein said therapeutic agent comprises a chemotherapeutic agent.

49. The composition of claim 48, wherein said conjugate comprises a pharmacologically active form of said chemotherapeutic agent.

50. The composition of claim 48, wherein said conjugate comprises a prodrug form of said chemotherapeutic agent.

51. The composition of claim 48, wherein said near-infrared dye and said chemotherapeutic agent are covalently bound via a linker.

52-82. (canceled)
